(19) United States
(12) Patent Application Publication
(10) Pub. No.: US 2026/0290609 A1
(43) Pub. Date: Sep. 24, 2026

(54) SYSTEM

(71) Applicant: SoftBank Group Corp., Tokyo (JP)

(72) Inventor: Aimi HIROKAWA, Tokyo (JP)

(73) Assignee: SoftBank Group Corp., Tokyo (JP)

(21) Appl. No.: 19/564,272

(22) Filed: Mar. 12, 2026

(30) Foreign Application Priority Data
Mar. 19, 2025 (JP) 2025-044950

(51) Int. Cl. G16H 50/30; G06N 3/0475; G16H 50/20

(52) U.S. Cl. CPC G16H 50/30; G06N 3/0475; G16H 50/20

(57) ABSTRACT

A system includes a processor, the processor is configured to, receive a user voice using a microphone for voice input, convert acquired voice data into text data by using a speech recognition technique, generate a prompt by using a generative AI model in order to understand an intention of the user, execute a function corresponding to the intention of the user based on the prompt, extract a feature from the voice data to recognize an emotion of the user, and adjust content or a method of a provided service based on the recognized emotion.

### CROSS-REFERENCE TO RELATED APPLICATION

This application is based on and claims priority under 35 USC 119 from Japanese Patent Application No. 2025-044950 filed on Mar. 19, 2025, the disclosure of which is incorporated by reference herein.

### BACKGROUND

### Technical Field

The Present Disclosure Relates to a System.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional voice-interaction systems such as voice assistants generally focus on converting voice input into text and mapping the text to predetermined functions. Such systems are often limited to simple command recognition and do not sufficiently understand the user's deeper intention, nor do they flexibly adapt the provided services to the user's emotional state.

In particular, existing systems typically rely on fixed rule-based or intent-classification logic that is not capable of dynamically generating complex prompts or instructions to interpret nuanced user needs. As a result, when the user expresses a complicated request, uses ambiguous expressions, or combines multiple intentions in a single utterance, the system fails to provide an appropriate response or sufficient support.

Furthermore, conventional systems usually do not perform emotional analysis based on acoustic features of the voice itself. Even when sentiment is estimated from text, such estimation is often inaccurate in scenarios where tone, pitch, or other prosodic elements strongly influence the user's emotional state. Therefore, conventional systems are unable to appropriately change the content or method of service provision based on the user's real-time emotional condition.

These limitations are particularly problematic in applications that require personalized and sensitive support, for example, health condition monitoring and the management of delicate information such as ending notes and wills, including digital assets. In such fields, the system is required not only to understand the literal content of the user's speech but also to infer the user's intention and to respond in a manner that takes into account the user's emotional state such as anxiety, stress, or relief.

Accordingly, there is a need for a system that can (i) accurately convert user voice into text, (ii) generate prompts by using a generative AI model in order to deeply understand the user's intention, (iii) execute functions corresponding to the understood intention, and (iv) extract features from voice data to recognize the user's emotion and adjust the content and method of the provided service based on the recognized emotion, thereby improving user experience and reliability in fields such as health management and will or ending-note support.

### SUMMARY

In order to solve the above-described problems, an aspect of the present invention provides a system comprising a processor, wherein the processor is configured to receive a user voice using a microphone for voice input, convert acquired voice data into text data by using a speech recognition technique, generate a prompt by using a generative AI model in order to understand an intention of the user, execute a function corresponding to the intention of the user based on the prompt, extract a feature from the voice data to recognize an emotion of the user, and adjust content or a method of a provided service based on the recognized emotion.

In the system according to this aspect, the processor uses the microphone to acquire the user's voice and applies a speech recognition technique so that the voice data can be converted into text data. By using the converted text data, the processor generates, by means of a generative AI model, a prompt that can represent or expand the user's request in a form that is suitable for downstream processing. The generative AI model allows the processor to flexibly interpret ambiguous or complex user expressions and to understand the underlying intention of the user beyond simple keyword matching.

The processor then executes a function that corresponds to the understood intention of the user based on the generated prompt. For example, in a case where the user's intention is related to health condition monitoring, the processor may start a health management function that acquires or calculates health-related parameters and provides health management information to the user. In a case where the user's intention is related to managing personal documents such as an ending note or a will, the processor may start a function that supports creation, update, and management of such documents including digital assets.

Additionally, the processor extracts acoustic features from the voice data, such as pitch, intensity, spectral characteristics, and temporal variations, in order to recognize the emotional state of the user. The processor performs emotion recognition based on these acoustic features either alone or in combination with the text data. The processor then adjusts the content or the method of service provision in accordance with the recognized emotion. For instance, when the user is recognized as anxious or distressed, the processor may adjust the wording, tone (in the case of synthesized speech), or level of detail of explanations, and may provide more careful and stepwise guidance.

According to another aspect of the present invention, the processor is configured to monitor a health condition of the user and provide health management information. By combining the above-described intention understanding and emotion recognition functions with health monitoring, the system can provide health information and advice that are tailored to the user's current emotional state and expressed needs, thereby supporting continuous and user-friendly health management.

According to still another aspect of the present invention, the processor is configured to support creation of an ending note or a will of the user and manage the ending note or the will including digital assets. In this aspect, the system can guide the user, through voice-based interaction, in preparing and updating sensitive documents and managing related digital assets. By utilizing generative AI-based prompt generation and emotion recognition, the system can adapt explanations and questions to the user's emotional condition and comprehension level, thereby offering more appropriate support in emotionally sensitive processes such as will creation and management of digital legacies.

The term “processor” refers to a hardware component or a combination of hardware and software components that executes instructions to perform data processing, control, and communication operations within the system.

The term “microphone” refers to a hardware input device configured to convert sound waves, including a user's spoken voice, into electrical or digital signals that can be processed by the processor.

The term “voice data” refers to digital data representing an audio signal of a user's spoken voice acquired through the microphone.

The term “text data” refers to character-based digital data obtained by converting voice data into a textual representation of the user's speech.

The term “speech recognition technique” refers to a software-implemented or hardware-implemented algorithm, model, or service that analyzes voice data and outputs corresponding text data representing the recognized speech content.

The term “generative AI model” refers to a machine learning model, such as a large language model or other generative model, that is trained to generate text or other outputs based on input data, and that is capable of producing a prompt or instruction sequence for interpreting a user's intention.

The term “prompt” refers to text or structured data generated by the generative AI model and used as an internal representation or instruction set to interpret, clarify, or expand the user's request and to determine a corresponding function to be executed.

The term “user's intention” refers to a purpose, request, or desired operation implied or explicitly stated by the user through voice input, including but not limited to requests related to health management, ending-note creation, or will management.

The term “function corresponding to the intention of the user” refers to a software-implemented processing operation or set of operations that is selected and executed based on the interpreted user's intention, such as health monitoring, information provision, document creation, or data management.

The term “feature” refers to a numerical or symbolic representation extracted from voice data, including but not limited to acoustic parameters such as pitch, energy, spectral characteristics, and temporal patterns, which is used for emotion recognition or other analysis.

The term “emotion of the user” refers to an emotional state of the user, such as happiness, sadness, anxiety, calmness, or anger, inferred by analyzing features extracted from the user's voice data, optionally in combination with text data.

The term “content of a provided service” refers to the substantive information, messages, instructions, advice, or documents that the system outputs or presents to the user in response to the user's intention.

The term “method of a provided service” refers to the manner, style, sequence, or modality in which the system delivers the content of a provided service, including but not limited to the level of detail, tone of expression, pacing, and choice of interaction channels such as text display or speech output.

The term “health condition of the user” refers to a physical or mental state of the user, including but not limited to vital signs, activity levels, health history, or other health-related indicators that may be monitored or evaluated by the system.

The term “health management information” refers to information related to the user's health condition, including evaluations, warnings, recommendations, summaries, or guidance provided to support health maintenance, improvement, or medical consultation.

The term “ending note” refers to a document or data set prepared by the user to record wishes, instructions, personal information, or other matters to be referenced in the event of serious illness, death, or incapacity, and which may include non-legally binding but practically important information.

The term “will” refers to a document that expresses the user's testamentary intentions regarding distribution of assets and other matters after death, and that is intended to have legal effect in accordance with applicable laws and regulations.

The term “digital assets” refers to electronically stored or managed assets associated with the user, including but not limited to online accounts, cryptocurrencies, digital files, subscription rights, digital media, and other forms of property or rights existing in digital form.

The term “manage the ending note or the will including digital assets” refers to operations performed by the system to create, store, update, organize, present, or control access to the ending note or will and associated digital assets, in accordance with the user's instructions and applicable security or privacy settings.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

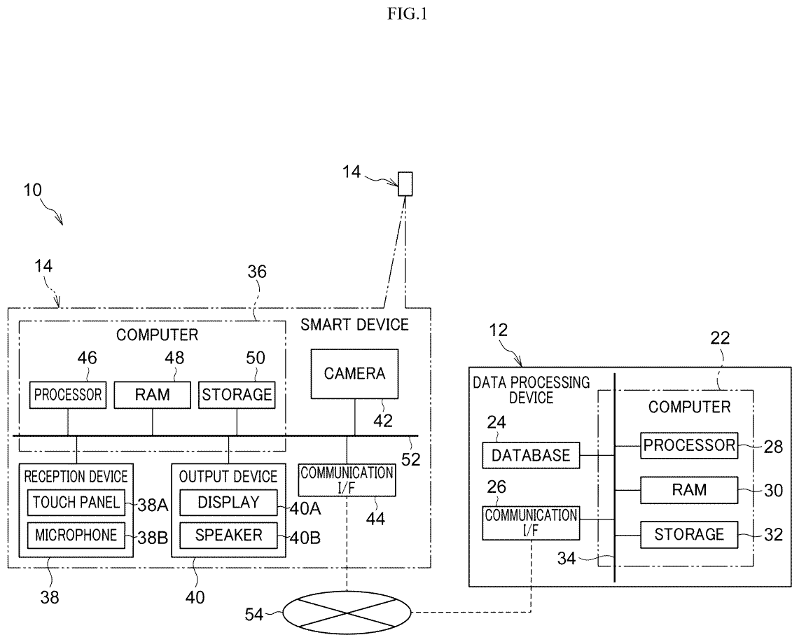

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

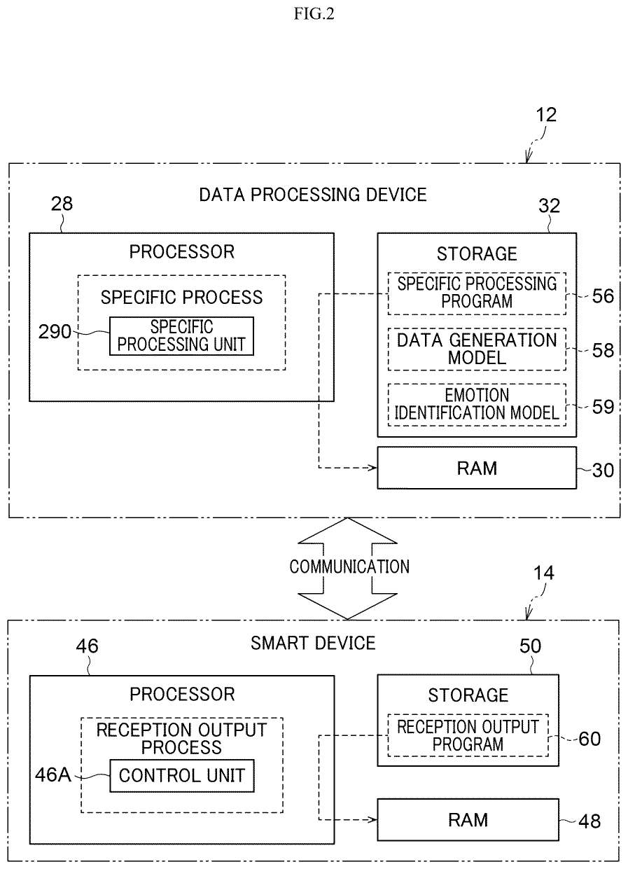

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

In conventional voice-based user interfaces and service provision systems, multiple independent components are typically used for speech recognition, intent understanding, data retrieval, and response generation. These components are often loosely coupled and rely on static rules, fixed prompts, or manually designed dialogue flows. As a result, several technical problems arise in terms of computer technology.

First, a conventional processor that simply converts speech to text and applies basic pattern matching or rule-based intent classification often fails to robustly interpret diverse and ambiguous user utterances. This leads to frequent misclassification of user intent, fragmented task execution, and the need for repeated user input. From a computational perspective, the processing pipeline is not optimized to leverage context, user state, and generated language in an integrated manner, which results in inefficient utilization of processing resources and network calls.

Second, known systems generally do not dynamically adapt internal processing or output generation based on user state information such as health-related activity data or emotional state inferred from acoustic features. Even where some form of personalization is attempted, it is implemented as a separate post-processing layer, and the underlying computing pipeline—speech recognition, natural language processing, and service invocation—remains insensitive to user state. Consequently, the processor executes generic processing flows that are not optimized for the individual user's context, leading to redundant computations, suboptimal network interactions, and increased latency for personalized content.

Third, integration of generative artificial intelligence models into conventional systems is often ad hoc. Prompt sentences are typically hard-coded or manually authored, without systematic generation based on structured intent information, state information, or external environment information. This results in unstable quality and low controllability of the generated outputs, as the computing device cannot automatically tailor prompts to current context. Accordingly, the processor may need to perform multiple iterations of generation, filtering, or post-processing, thereby increasing computational load, network traffic, and response time.

Fourth, conventional architectures do not provide a unified mechanism for combining (i) analytical processing of structured data obtained from external information services or state information management services, and (ii) generative natural-language processing. Structured data such as activity logs, biometric metrics, document templates, and asset lists are typically processed in separate subsystems and only loosely integrated with generated text. This separation forces the computer system to maintain multiple data representations and transformation layers, increasing memory consumption and processing overhead, and complicating error handling across modules.

Fifth, emotion recognition from voice, where implemented, is often used only for superficial user interface changes such as changing colors or playing different sounds, and is not integrated into the core decision-making pipeline. The processor does not change the internal prompt construction, service selection, or output structure based on the estimated emotional state, which limits the capability of the computing device to adapt its processing strategy and communication style in real time. As a result, the overall interaction loop between the user and the computer system remains rigid and requires additional user effort to clarify requests or correct misunderstandings.

Accordingly, there is a need for a system and processor configuration that technically improves the way a computer acquires voice input, interprets user intent, generates prompts for a generative information processing model, integrates user state and external environment information, and dynamically adjusts both internal processing and output expression based on an estimated emotional state. The technical problem is to provide an improved computer-implemented architecture that reduces misinterpretation of user intent, reduces redundant computation and network access, enhances the efficiency and reliability of interaction flows, and generates contextually appropriate outputs by tightly integrating recognition, analysis, generation, and state-aware adaptation within a single processing pipeline.

The specific processing by the specific processing unit 290 of the data processing device 12 in Example 1 is realized by the following means.

The present invention provides a server comprising at least one processor and at least one memory storing instructions that, when executed by the at least one processor, cause the server to cooperate with a terminal device to acquire an acoustic signal of a user by using a voice acquisition device of the terminal device, convert the acquired acoustic signal into digital data and input the digital data to a speech recognition function to obtain character information, input the character information to a natural language processing function to perform morphological analysis, syntactic analysis, and semantic analysis and to extract request information and intent information indicating a request content and an intention of the user, generate a prompt sentence for a generative information processing model based on input information including the intent information and the character information, input the prompt sentence to the generative information processing model and obtain, from the generative information processing model, processing content or output information corresponding to the intention of the user, select, based on the intent information, at least one of an external information acquisition function and a state information acquisition function configured to acquire user state information or external environment information, access, via a network, an external information processing service or a state information management service to acquire the user state information or the external environment information, perform at least one of an aggregation process, a comparison process, and an evaluation process on the acquired user state information or the external environment information and combine a result of the at least one process with the output information obtained from the generative information processing model to generate service content or proposal content, extract acoustic feature values from the acoustic signal and input the acoustic feature values to an emotion estimation model to estimate an emotional state of the user, change at least one of an expression form and a providing method of the prompt sentence, the service content, or the proposal content in accordance with the emotional state, and cause the terminal device to present the service content or the proposal content to the user and to acquire additional voice input from the user for iterative processing. This enables a computer system to technically improve end-to-end voice-based interaction by tightly integrating speech recognition, natural language understanding, state-aware prompt generation for a generative model, structured data evaluation, and emotion-based adaptation into a unified processing pipeline, thereby reducing misinterpretation of user intent, minimizing redundant processing and network access, and generating contextually appropriate and personalized outputs with increased computational efficiency and responsiveness.

The term “acoustic signal” refers to an analog or digital representation of sound produced by a user, including voice utterances, that is capable of being captured by a voice acquisition device and processed by an information processing apparatus.

The term “voice acquisition device” refers to a hardware component or combination of hardware components, such as a microphone and associated circuitry, that is configured to capture an acoustic signal from a user and convert the acoustic signal into an electrical or digital signal usable by a processor.

The term “digital data” refers to numerical data obtained by sampling and quantizing an analog acoustic signal in a time domain or a frequency domain, the numerical data being suitable for processing by a speech recognition function or other software modules.

The term “speech recognition function” refers to a software-based or hardware-based processing function that receives digital data representing an acoustic signal and outputs character information corresponding to a linguistic transcription of the acoustic signal.

The term “character information” refers to text data composed of one or more characters, symbols, or code points representing words or sentences derived from an acoustic signal of a user.

The term “natural language processing function” refers to a software-based or hardware-based processing function that performs at least one of morphological analysis, syntactic analysis, semantic analysis, named entity recognition, or intent classification on character information.

The term “request information” refers to structured data indicating a type of operation, service, or task requested by a user, the structured data being derived from character information by the natural language processing function.

The term “intent information” refers to structured data representing an inferred intention, goal, or purpose of a user, extracted from character information by the natural language processing function and used to control subsequent processing.

The term “prompt sentence” refers to a text sequence or data structure generated on the basis of at least the intent information and the character information, the text sequence or data structure being configured as an input to a generative information processing model to control behavior of the generative information processing model.

The term “generative information processing model” refers to a machine-learned model, such as a neural network model, that generates output information including natural language text or other data by processing an input including a prompt sentence.

The term “processing content” refers to a description of operations, actions, or procedures to be performed by a system or service, the description being generated by the generative information processing model in response to a prompt sentence.

The term “output information” refers to data produced by a generative information processing model, including at least one of natural language text, structured data, control parameters, or recommendations, corresponding to an intention or request of a user.

The term “external information acquisition function” refers to program logic that selects and invokes one or more external information processing services via a network to obtain external environment information or other data relevant to a user's request.

The term “state information acquisition function” refers to program logic that selects and invokes one or more services or modules configured to manage or provide user state information, such as physiological or behavioral data, to be used in subsequent processing.

The term “user state information” refers to information indicative of a condition or status of a user, including but not limited to biological activity information, biometric information, behavioral information, or historical interaction information.

The term “external environment information” refers to information indicative of conditions or states external to a user and a terminal device, including but not limited to environmental data, service data, or resource data obtained from external information processing services.

The term “external information processing service” refers to a computing service accessible via a network, which receives a request from a system and returns external environment information or other processed data.

The term “state information management service” refers to a computing service or module that stores, aggregates, or manages user state information and provides such information in response to a request from a system.

The term “aggregation process” refers to a computational process that combines multiple pieces of user state information or external environment information, for example by summation, averaging, or grouping, to produce aggregated information.

The term “comparison process” refers to a computational process that compares user state information or external environment information with one or more reference values, thresholds, historical values, or target values.

The term “evaluation process” refers to a computational process that assesses user state information or external environment information according to predetermined criteria, rules, or models, to produce evaluation results indicative of a condition or performance level.

The term “service content” refers to information, guidance, or actions generated by the system and intended to be provided to a user as a service, including at least one of explanatory text, advice text, recommendations, or control instructions.

The term “proposal content” refers to information generated by the system and intended to propose actions, plans, or changes to a user, including recommendations, options, or alternative scenarios.

The term “acoustic feature values” refers to numerical descriptors calculated from an acoustic signal, including but not limited to energy, pitch, formant, spectral, prosodic, or temporal features, which are used as input to an emotion estimation model.

The term “emotion estimation model” refers to a machine-learned model or rule-based model that receives acoustic feature values or other input and outputs an estimated emotional state of a user.

The term “emotional state” refers to a state of affect or emotion of a user, such as happiness, sadness, anger, calmness, or stress level, represented as a discrete category, continuous value, or multi-dimensional vector.

The term “expression form” refers to a style, tone, structure, or format of text or other output information presented to a user, including but not limited to politeness level, length, complexity, or emotional nuance.

The term “providing method” refers to a manner in which service content or proposal content is presented to a user, including selection among modalities such as visual display, audio output, or combined modes, and timing or frequency of presentation.

The term “display device” refers to a hardware component, such as a screen or monitor, that is configured to visually present information including service content or proposal content to a user.

The term “audio output device” refers to a hardware component, such as a speaker or earphone, that is configured to output sound representing service content, proposal content, or other audio information to a user.

The term “biological activity information” refers to data indicative of physical activities or physiological signals of a user, including but not limited to activity amount information, heart rate information, and sleep information.

The term “activity amount information” refers to data representing a quantity or intensity of physical activity of a user over time, such as step count, movement distance, or exercise duration.

The term “heart rate information” refers to data representing a cardiac activity of a user, including instantaneous heart rate, average heart rate, or heart rate variability.

The term “sleep information” refers to data representing sleep-related behavior of a user, including sleep duration, sleep stages, or sleep interruptions.

The term “document template information” refers to structured data defining a layout, sections, or placeholders for generating a document, such as a record document or an expression-of-intention document.

The term “asset information” refers to data representing properties, rights, or resources associated with a user, including but not limited to financial assets, physical assets, or digital assets.

The term “document type” refers to a classification of a document according to its purpose or structure, such as a record document, a contract document, or an expression-of-intention document.

The term “asset type” refers to a classification of an asset according to its category or characteristics, such as a financial asset, a physical asset, or a digital asset.

The term “record document” refers to a document that records information relating to a user's status, preferences, or plans, including but not limited to logs, notes, or preparation documents.

The term “expression-of-intention document” refers to a document that explicitly states a user's wishes, instructions, or decisions regarding future actions, disposition of assets, or personal matters.

The term “end-of-life preparation” refers to planning activities of a user related to matters occurring near or after the end of the user's life, including management of assets, personal messages, and instructions for successors.

The term “terminal device” refers to a user-operated computing device, such as a mobile device, a tablet device, or a personal computer, that is configured to interact with a server and provide input and output for a user.

The term “information processing apparatus” refers to one or more computing devices including at least one processor and memory, configured to execute programs for processing acoustic signals, character information, and other data.

The term “server” refers to an information processing apparatus or a group of information processing apparatuses that provide computation, storage, or services to one or more terminal devices via a network.

In one embodiment, a server cooperates with a terminal to implement the claimed system. The terminal includes a microphone, a speaker, a display, a network interface, and a local processor with a memory. The server includes at least one central processing unit (CPU), at least one graphics processing unit (GPU) or tensor processing unit (TPU), a main memory, and a persistent storage device. The server also includes a network interface for communication with a plurality of terminals over a packet-switched network.

The terminal acquires an acoustic signal of a user by using the microphone. The terminal converts the analog acoustic signal into digital data by means of an audio codec and an operating system audio driver. The terminal, or alternatively the server, applies a speech recognition function implemented by a sequence-to-sequence neural network, such as a recurrent neural network with long short-term memory units or a transformer-based acoustic-to-text model, to convert the digital data to character information. The character information is transferred between modules as a Unicode text string stored in a string buffer structure in memory.

The server uses a natural language processing function to process the character information. The natural language processing function includes a tokenizer, a part-of-speech tagger, a dependency parser, and a semantic role labeling module. The server represents the character information internally as a token sequence, where each token has associated tags such as lemma, part-of-speech, syntactic head index, and named entity type. The server computes request information and intent information by applying a classifier to a fixed-length vector embedding of the token sequence. The classifier may be implemented as a feedforward neural network or as a softmax layer on top of a pre-trained language representation model. The intent information is stored as a structured data object that includes an intent label and a set of key-value parameters.

The server generates a prompt sentence for a generative AI model based on the intent information and the character information. The server constructs the prompt sentence by applying a rule-based template engine that selects a prompt pattern according to the intent label and fills slots in the pattern with parameter values extracted from the intent information and with relevant parts of the original character information. The prompt sentence is maintained as a text buffer with explicit markers that control the behavior of the generative AI model. For example, when the user requests health-related advice, the server constructs a prompt sentence such as:
“The user's health data for today shows 5,000 steps completed out of an 8,000-step goal. Generate a friendly and concise message explaining this progress and suggesting simple actions to reach the goal.”

In another case, when the user requests document creation support, the server constructs a prompt sentence such as:
“The user wants to create a last will and testament. The user's name is John Doe. The beneficiaries are: 1) Jane Doe (spouse), 2) Alex Doe (child). The major assets include a house, a savings account, and a car. Draft a clear and structured will in English, using formal legal style. Include sections for appointment of executor, distribution of assets, and revocation of previous wills. Do not include jurisdiction-specific clauses.”

The server inputs the prompt sentence to a generative AI model. In one embodiment, the generative AI model is implemented as a transformer-based neural network with multiple self-attention layers, residual connections, and layer normalization, trained on large-scale text corpora. The server represents the prompt sentence as a sequence of token identifiers and corresponding positional encodings. The generative AI model computes a sequence of hidden state vectors, and then applies a softmax layer over a vocabulary to generate output tokens in an auto-regressive manner. The server decodes the output tokens into output information that may include natural language text describing processing content, explanations, or recommendations.

The server selects, on the basis of the intent information, an external information acquisition function or a state information acquisition function. The external information acquisition function uses a client module to access external information processing services via the network. For example, the server uses an application programming interface client to send an HTTP request to an environmental information service and receive structured external environment information in a serialized format. The state information acquisition function accesses a state information management service that stores user state information, such as biological activity information including steps, heart rate, and sleep duration. The server represents the user state information and the external environment information as time-series data structures, for example arrays of timestamp-value pairs.

The server applies an aggregation process by computing sums, averages, or weighted statistics over the arrays to obtain aggregated indicators. The server performs a comparison process by comparing the aggregated indicators with reference values or goals stored in a configuration database. The server executes an evaluation process by applying rule-based logic or a trained regression model to generate evaluation scores or categories. For example, the server computes a ratio of actual steps to target steps and assigns an evaluation category such as “below target,” “near target,” or “above target.” The server combines the evaluation results with the output information from the generative AI model to construct service content or proposal content, which is represented as a composite structure including both textual elements and numerical summaries.

The terminal, or the server acting on behalf of the terminal, extracts acoustic feature values from the acoustic signal. The feature extraction process computes, for example, Mel-frequency cepstral coefficients, pitch contours, energy envelopes, and temporal statistics over sliding windows of the digital audio data. The server inputs the acoustic feature values to an emotion estimation model, which may be implemented as a convolutional neural network or a recurrent neural network trained on labeled emotional speech data. The emotion estimation model outputs an emotional state label or a continuous emotional score. The server stores the emotional state together with a timestamp to be used in subsequent processing.

The server changes at least one of the expression form and the providing method of the prompt sentence, the service content, or the proposal content in accordance with the estimated emotional state. For example, the server selects a more encouraging and gentle wording style when the emotional state indicates stress or sadness, and uses shorter and more direct expressions when the emotional state indicates urgency. The server modifies the prompt sentence to the generative AI model by including explicit instructions about tone and length, such as “Use a calm and reassuring tone” or “Provide a brief and direct explanation.” In this manner, the emotional state directly influences the internal behavior of the generative AI model through systematic modifications of the prompt sentence, rather than only affecting superficial interface elements.

The terminal presents the service content or the proposal content to the user by displaying the text on the display and by optionally converting the text to speech using a text-to-speech module.

The terminal stores the presentation history and the associated intent information in a local cache to maintain session context. The user can provide additional voice input in response to the presented content, and the system reuses the existing context and state information in subsequent processing.

The server, by integrating speech recognition, natural language understanding, state-aware prompt generation, external data retrieval, structured data evaluation, and emotion-based adaptation in a coordinated architecture, improves core computer technology. Specifically, the server reduces redundant network requests by selecting external information acquisition functions only when the intent information indicates a need for external or state data. The server reduces misinterpretations by combining intent information with structured user state information and by reinforcing the interpretation through prompt sentences that encode context explicitly. The server improves processing efficiency by using shared data structures for character information, intent information, prompt sentences, and evaluation results, thereby avoiding repeated conversions and parsing operations.

The server improves accuracy of generated outputs by feeding the generative AI model with prompt sentences that include precise contextual and numerical information. This reduces the need for repeated model invocations and post-processing, and leads to faster convergence of the interaction. The server also reduces communication load by limiting the size and frequency of requests to external services based on the intent information and by caching aggregated results.

The explicit structuring of data, such as intent objects, time-series state information, and evaluation result objects, enables the server to perform incremental updates instead of recomputing entire analyses.

The generative AI model is not used as a black box; rather, the server constrains and guides the model through systematically constructed prompt sentences. The server employs non-conventional rules for prompt generation, including insertion of machine-readable markers, explicit separation of user input and system instructions, and incorporation of structured data in controlled formats. These rules enable the generative AI model to produce outputs that can be reliably parsed and combined with other data structures, which is a technical improvement over systems that simply pass raw user queries to a model.

The emotion estimation model contributes to technical improvement by allowing the server to adapt processing paths. For example, when the emotional state indicates confusion, the server automatically selects a more detailed explanatory template and may reduce the amount of external data requested to avoid overloading the user. When the emotional state indicates confidence, the server selects a shorter and more aggregated output. This dynamic adaptation reduces unnecessary processing and data transfer, which leads to lower latency and reduced computational load.

The server trains the generative AI model and the emotion estimation model using supervised or self-supervised learning. During training, the server defines a loss function that may include cross-entropy loss for token prediction, auxiliary losses for intent consistency, and regularization terms to prevent overfitting. The server updates model parameters using gradient-based optimization, such as stochastic gradient descent or adaptive moment estimation. The server may apply data augmentation techniques, such as adding noise to acoustic signals or paraphrasing text, to increase robustness. By optimizing these models specifically for the integrated architecture, the server achieves better computational efficiency and higher accuracy than generic models not tuned for such usage.

In another embodiment, the terminal executes part or all of the natural language processing and emotion estimation locally, using a reduced-size model optimized for on-device execution. In this case, the server focuses on heavy computations, such as large generative AI model inference and large-scale state information aggregation. A hybrid configuration can be used in which the terminal performs preliminary intent detection to decide whether to invoke the server-side generative AI model, thereby reducing network usage and server load.

In yet another embodiment, the server supports multiple types of external information processing services and state information management services. The server maintains a registry of service endpoints, supported data types, and quality-of-service parameters. The server selects an appropriate service based on the intent information, user preferences, and historical performance metrics. For example, the server may select a more detailed health information service when the user repeatedly requests health-related explanations, and a simpler service when the user only requests summary information. This adaptive service selection further reduces latency and computational overhead.

In still another embodiment, the server manages different document template information and asset information structures for various document types. The server can generate record documents and expression-of-intention documents not only for end-of-life preparation, but also for other contexts, such as authorization letters or planning notes. The server uses the same underlying intent information and prompt generation mechanisms, but applies different template selection strategies and evaluation rules. This demonstrates that the technical contributions of the architecture are not limited to a particular business scenario, but relate to improvements in data handling, model invocation, and interaction control in computer systems.

Through these embodiments, the server and the terminal cooperate to implement a system in which generative AI models, prompt sentences, structured state and environment data, and emotion-aware adaptation are integrated in a way that enhances processing accuracy, reduces computational and communication overhead, and improves overall responsiveness. The system therefore provides a concrete improvement in computer technology, rather than merely automating a human workflow.

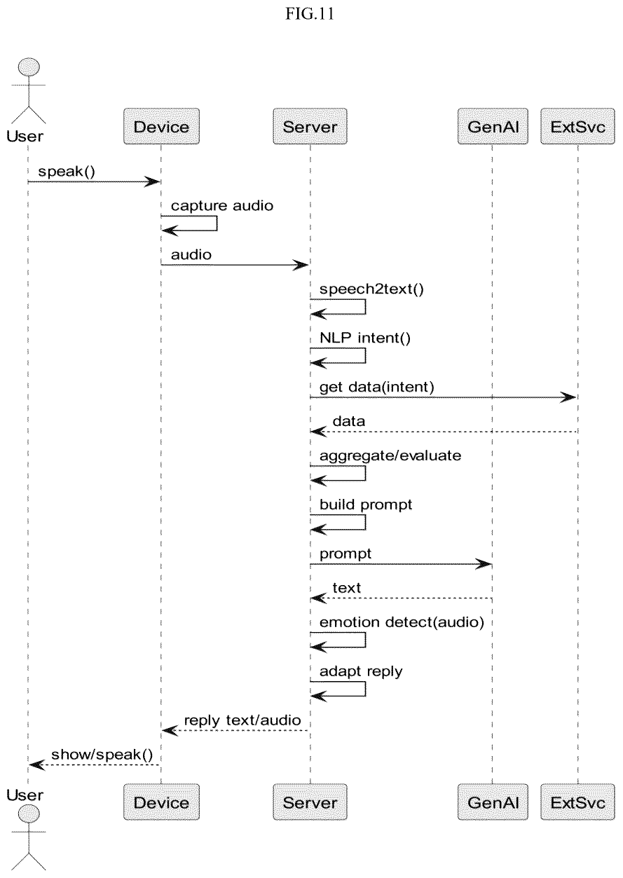

The following describes the processing flow using FIG. 11.

### Step 1:

The user provides a voice input.

The user speaks a request, such as “Check my health status” or “I want to create a will,” into the microphone of the terminal.
Input: No digital input; the user produces an acoustic signal.
Output: An analog acoustic signal in air near the terminal.

### Step 2:

The terminal acquires the acoustic signal and converts it to digital audio data.

The terminal uses the microphone and an audio driver to sample the acoustic signal at a predetermined sampling rate (for example, 16 kHz, 16-bit mono). The terminal converts the analog waveform to a sequence of pulse-code modulation samples and stores the samples into an audio buffer in memory until the user finishes speaking. The terminal may apply voice activity detection to determine the start and end of the utterance.
Input: Analog acoustic signal from the user.
Output: A sequence of digital audio samples representing the user's utterance.

### Step 3:

The terminal sends the digital audio data for speech recognition and receives character information.

The terminal packages the digital audio samples and metadata (sampling rate, encoding format, language code) and sends them to a speech recognition function, which may run locally or on the server. The speech recognition function performs feature extraction (for example, Mel-frequency cepstral coefficients), feeds the extracted features into an acoustic-language model, and decodes the most probable text sequence. The terminal receives the decoded text and stores it as a Unicode string.
Input: Digital audio samples representing the user's utterance.
Output: Character information representing the recognized text of the user's utterance.

### Step 4:

The server performs natural language processing to derive request information and intent information.

The server receives the character information from the terminal. The server tokenizes the text into words, assigns part-of-speech tags, and performs syntactic and semantic analysis. The server then embeds the token sequence into a vector representation and applies an intent classification model to determine an intent label (for example, “health_status_check” or “create_document”).

The server also extracts parameters such as time expressions, document types, or asset categories.

The server stores the result as request information and intent information in a structured object.
Input: Character information representing the recognized user utterance.
Output: Request information and intent information, including an intent label and associated parameters.

### Step 5:

The server selects an information acquisition function based on the intent information.

The server examines the intent label and parameters to determine whether user state information or external environment information is required. If the intent is related to health evaluation, the server selects a state information acquisition function; if the intent is related to external conditions, such as weather, the server selects an external information acquisition function. The server maps each intent label to a specific acquisition function identifier and stores this selection for the next processing step.
Input: Intent information including the intent label and parameters.
Output: A selected acquisition function identifier indicating a state information acquisition function or an external information acquisition function.

### Step 6:

The server acquires user state information or external environment information.

The server uses the selected acquisition function to call corresponding external services or internal modules. For user state information, the server sends authenticated requests to a state information management service and receives data such as step counts, heart rate, or sleep duration. For external environment information, the server sends requests to external information processing services and receives data such as weather forecasts or other environmental metrics.

The server parses the responses and stores them as time-series data or structured records.
Input: Acquisition function identifier, intent information, and user identification or context.
Output: User state information and/or external environment information in structured form.

### Step 7:

The server performs aggregation, comparison, and evaluation on the acquired information.

The server aggregates the time-series data by computing sums, averages, or other statistical values over a specified period. The server compares these aggregated values with reference values or goals stored in a configuration store, and then evaluates the user's status according to predetermined rules. For example, the server computes the ratio of actual steps to a target step count and classifies the ratio into categories. The server outputs evaluation results that summarize the condition or performance of the user or environment.
Input: User state information and/or external environment information, and reference values or goals.
Output: Evaluation results, including aggregated indicators and classification or scoring information.

### Step 8:

The server generates a prompt sentence for a generative AI model.

The server constructs a prompt sentence by selecting a template associated with the intent label and filling template slots with the character information, the intent parameters, and the evaluation results. The server may adjust wording and structure of the prompt sentence to control length, style, and level of detail. For a health-related request, the server may generate a prompt sentence such as:
“The user's health data for today shows 5,000 steps completed out of an 8,000-step goal. Generate a friendly and concise message explaining this progress and suggesting simple actions to reach the goal.”

For a document-related request, the server may generate a prompt sentence such as:
“The user wants to create a last will and testament. The user's name is John Doe. The beneficiaries are: 1) Jane Doe (spouse), 2) Alex Doe (child). The major assets include a house, a savings account, and a car. Draft a clear and structured will in English, using formal legal style. Include sections for appointment of executor, distribution of assets, and revocation of previous wills. Do not include jurisdiction-specific clauses.”
Input: Character information, intent information, and evaluation results.
Output: A prompt sentence constructed as a text string for input to a generative AI model.

### Step 9:

The server generates output information using a generative AI model.

The server encodes the prompt sentence into a sequence of tokens and feeds the tokens into a generative AI model. The model computes hidden representations and outputs a sequence of tokens corresponding to generated text. The server decodes the tokens into natural language and may perform light post-processing, such as trimming unwanted text or normalizing punctuation.

The server then stores the generated output information as a text string or a structured document fragment.
Input: Prompt sentence encoded as a token sequence.
Output: Output information generated by the generative AI model, including natural language text corresponding to the user's intent.

### Step 10:

The terminal or the server extracts acoustic feature values from the original acoustic signal.

The terminal or the server takes the digital audio samples from Step 2 and segments them into short frames. The processor computes acoustic feature values such as energy, pitch, spectral coefficients, and temporal derivatives for each frame. These feature values are combined into a multidimensional feature vector sequence and normalized across the utterance.
Input: Digital audio samples representing the user's utterance.
Output: Acoustic feature values organized as a sequence of feature vectors.

### Step 11:

The server estimates an emotional state of the user based on the acoustic feature values.

The server feeds the sequence of acoustic feature vectors into an emotion estimation model. The model processes the sequence and outputs either probabilities for discrete emotion categories or continuous scores for emotional dimensions. The server selects the most likely emotion category or constructs an emotional state descriptor from the scores. The server then stores this emotional state as context information associated with the corresponding utterance.
Input: Acoustic feature values representing the user's utterance.
Output: An estimated emotional state describing the user's affective condition.

### Step 12:

The server adapts the prompt sentence and generated content based on the emotional state.

The server analyzes the estimated emotional state and chooses adaptation rules. The server may regenerate or modify the prompt sentence by inserting instructions about tone and style, such as “Use a calm and reassuring tone” when the user appears stressed. The server may also adjust the length and complexity of the output information by selecting between different templates or regeneration strategies. The server updates the service content or proposal content to match the emotional state before sending it to the terminal.
Input: Original prompt sentence, output information, and emotional state.
Output: Adapted prompt sentence and adapted service content or proposal content aligned with the emotional state.

### Step 13:

The terminal presents the service content or proposal content and accepts additional voice input.

The terminal receives the adapted service content or proposal content from the server. The terminal displays the text on the display and may convert the text to speech using a text-to-speech engine, outputting audio through the speaker. The user views or listens to the presented content and, if desired, provides additional voice input to refine the request or ask follow-up questions.

The terminal captures the additional voice input as a new acoustic signal, which becomes the new input for repeating the processing flow.
Input: Adapted service content or proposal content from the server.
Output: Presented information to the user and, optionally, a new acoustic signal corresponding to additional user input.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional dialog systems that accept voice input and return information to users are typically implemented as a simple pipeline in which speech is transcribed into text, the text is matched against predefined commands, and a fixed response is returned. Such systems suffer from several technical limitations when deployed on general-purpose information processing hardware.

First, existing systems often treat speech recognition, natural language understanding, database search, and response generation as separate, loosely coupled components. As a result, intermediate representations, such as recognized text or search parameters, are not consistently structured and are not reused across different functional domains. This leads to redundant parsing and transformation steps, increased processing latency, and higher consumption of processing resources and network bandwidth on the server side.

Second, many systems are designed for a single domain, such as product search, health advice, or financial planning. When these domain-specific modules are simply aggregated, the server must maintain separate logic, separate data models, and separate user interfaces. This fragmentation prevents the processor from efficiently classifying the requested domain, constructing shared structured data, and reusing the same computational pathway. Consequently, the system tends to scale poorly as additional services are added, and the hardware utilization becomes inefficient.

Third, conventional systems generally do not integrate a generative AI model in a technically optimized way. Responses are often generated by directly feeding raw user text to a generative model, without providing structured data extracted from backend databases or state evaluation calculations. This causes the generative model to perform implicit information extraction or inference that has already been done elsewhere in the system, which wastes computational cycles on accelerator hardware and can degrade the accuracy and consistency between database outputs and generated explanations.

Fourth, existing architectures typically ignore or underutilize paralinguistic information, such as emotional state inferred from the user's voice signal. As a result, the system returns the same style of responses regardless of whether the user is confused, frustrated, or calm. This limits the practical usability of the system and often forces users to repeat queries or abandon the interaction, which increases the number of processing cycles and network round trips required to complete a task.

Fifth, when the same infrastructure is used to support multiple complex scenarios—such as in-store product navigation, longitudinal health monitoring, asset projection, and document drafting—there is no unified mechanism to generate prompt sentences that systematically combine recognized text, structured data, and system state into inputs for a generative AI model. The absence of such a mechanism hinders the server's ability to produce consistent, context-aware prompts, leading to unstable output quality and unnecessary recomputation on the server and accelerator hardware.

Accordingly, there is a need for an improved computer-implemented system and processing method in which a processor can (i) unify voice acquisition, text conversion, intent and domain classification, structured data generation, and call control to a generative AI model; (ii) dynamically adapt the contents and style of prompts and responses based on an estimated emotional state and a requested domain; and (iii) reuse a common processing framework across multiple service domains, including product information provision, health condition support, asset condition support, and document creation support. Such an improvement should reduce redundant computation, lower end-to-end latency, improve resource utilization on servers and accelerators, and increase the robustness and responsiveness of the overall dialog system.

The specific processing by the specific processing unit 290 of the data processing device 12 in Application Example 1 is realized by the following means.

The present invention provides a server comprising a processor and at least one storage device, wherein the processor is configured to acquire a voice signal of a user by using a voice input device, convert the acquired voice signal into character information by using a speech recognition technique, analyze input information including the character information and user attribute information to determine an intention of the user and a requested domain, generate, based on a result of the determination, processing request information representing at least one of product information provision, health condition support, asset condition support, and document creation support, the processing request information including search condition information or state evaluation information, search, based on the search condition information, a product information storage device and generate structured data by using product information acquired as a search result, acquire, based on the state evaluation information, related information from at least one of a health information storage device and an asset information storage device and generate structured data by using the acquired information, generate a prompt sentence including the character information and the structured data and input the prompt sentence into a generative AI model to cause the generative AI model to generate explanation information or recommendation information, integrate the generated explanation information or recommendation information with the structured data to generate response information to be presented to the user, extract features from the voice signal of the user and estimate an emotional state of the user based on the extracted features, dynamically adjust at least one of contents of the prompt sentence, an expression style of the explanation information or the recommendation information, and a presentation method of the response information in accordance with the emotional state and the requested domain, acquire, when the server is used in a physical facility that stores products, location information associated with the product information and add guidance information for navigation within the facility based on the location information to the response information, and repeat the processing in response to additional voice input from the user while updating the prompt sentence and the structured data in consideration of a dialogue history. This enables a unified and resource-efficient dialog processing architecture in which the same server-side computational pipeline converts raw voice input into structured data, optimized prompt sentences, and adaptive responses across multiple domains, thereby reducing redundant computation, lowering latency, improving utilization of general-purpose processors and accelerator hardware, and enhancing the technical performance and responsiveness of the overall computer system.

The term “voice input device” refers to an input apparatus, such as a microphone or an array of microphones, that converts sound waves produced by a user into an electrical or digital signal suitable for processing by an information processing device.

The term “voice signal” refers to a time-varying electrical or digital representation of acoustic sound produced by a user, which is obtained from a voice input device and used for subsequent speech, emotion, or feature analysis.

The term “character information” refers to symbolic textual data representing linguistic content recognized from a voice signal, including at least characters, words, or punctuation in a natural language.

The term “speech recognition technique” refers to a software-implemented algorithm or model that converts a voice signal into character information, and may include acoustic modeling, language modeling, and decoding processes executed on a processor.

The term “user attribute information” refers to information associated with a user, such as age range, preferred language, service usage history, domain preferences, or access permissions, which is stored in a storage device and used to contextualize processing of the user's requests.

The term “intention of the user” refers to a semantic objective or purpose underlying the user's input, such as requesting product information, seeking health advice, obtaining asset information, or requesting assistance in creating a document.

The term “requested domain” refers to a classification category indicating a technical or service area relevant to a user's request, including at least a product information domain, a health condition support domain, an asset condition support domain, or a document creation support domain.

The term “processing request information” refers to data generated by the processor that formally represents a user's intention and requested domain and includes one or more parameters, conditions, or identifiers used to control subsequent processing steps.

The term “search condition information” refers to structured parameters, such as category identifiers, price limits, quality thresholds, or keyword constraints, that define a query for retrieving information from a storage device.

The term “state evaluation information” refers to data representing a computed or estimated condition of the user or a related context, including at least health indices, activity indices, financial indices, or status metrics derived from stored information.

The term “product information storage device” refers to a memory or database system that stores records related to tangible or intangible items, including attributes such as item name, category, specification, price, availability, and location information.

The term “health information storage device” refers to a memory or database system that stores health-related data for a user, including biometric measurements, activity logs, lifestyle records, and derived health indices.

The term “asset information storage device” refers to a memory or database system that stores financial or asset-related data for a user, including balances, holdings, transaction records, and projected values of physical or digital assets.

The term “structured data” refers to data organized according to a predefined schema or format, such as key-value pairs, records, or tables, that allows programmatic access, search, and transformation by a processor.

The term “prompt sentence” refers to a text sequence or message generated by the processor that is formatted to be input into a generative AI model, and that includes at least recognized character information, structured data, and instructions indicating a desired type or style of generated output.

The term “generative AI model” refers to a parameterized computational model, such as a neural network-based language model, that receives a prompt sentence as input and generates new text or other data as output based on learned patterns.

The term “explanation information” refers to generated text or data that describes, clarifies, or summarizes underlying structured data, processing results, or system states in a human-understandable manner.

The term “recommendation information” refers to generated text or data that proposes one or more options, items, actions, or plans to a user, based on structured data, evaluation results, or preferences.

The term “response information” refers to information generated by the processor, including at least explanation information, recommendation information, and associated structured data, that is formatted for presentation to the user via an output device.

The term “features” refers to numerical or symbolic values extracted from a voice signal or other raw data, such as pitch, energy, spectral characteristics, prosody, or derived statistical descriptors, which are used as inputs to classification or estimation algorithms.

The term “emotional state” refers to an estimated affective condition of a user, such as calmness, happiness, confusion, or frustration, which is inferred by analyzing features extracted from a voice signal or other behavioral data.

The term “contents of the prompt sentence” refers to constituent elements included in a prompt sentence, such as user text, structured data summaries, contextual instructions, and style constraints provided to a generative AI model.

The term “expression style” refers to a manner in which information is expressed in generated text, including tone, formality level, length, degree of detail, and linguistic complexity.

The term “presentation method” refers to a mode or format for outputting response information to the user, including at least text display, audio output, graphical highlighting, ordering of items, or inclusion of navigation cues.

The term “physical facility that stores products” refers to a tangible environment such as a retail store, warehouse, or showroom, in which physical items are arranged at specific locations that can be described by location information.

The term “location information” refers to data indicating a position of an item or area within a physical facility, including at least identifiers of zones, aisles, shelves, coordinates, or directions.

The term “guidance information for navigation within the facility” refers to instructions, maps, or indicators generated by the processor that assist a user in moving from a current position to a target position within a physical facility, based on location information.

The term “dialogue history” refers to stored records of prior interactions between a user and the system, including past user inputs, system responses, and associated structured data and metadata, which are referenced in subsequent processing.

The term “biometric information” refers to measurable physical or physiological data of a user, such as heart rate, blood pressure, activity intensity, body temperature, or similar parameters.

The term “behavior information” refers to data describing actions or patterns of activity of a user, such as movement logs, usage frequency of services, or time-based behavior records.

The term “health index” refers to a numerical or categorical value computed from biometric information and behavior information that represents a summarized condition of a user's health status.

The term “lifestyle guidance information” refers to explanation information or recommendation information that suggests behavioral changes, habits, or routines in areas such as exercise, sleep, or nutrition, based on a health index.

The term “family structure information” refers to data describing relationships and identities within a user's family or household, such as roles, member identifiers, or basic attributes of related persons.

The term “wish information” refers to data indicating preferences, intentions, or desired outcomes expressed by a user, including instructions regarding distribution of assets, care after end-of-life, or other personal directives.

The term “asset-related information” refers to data associated with ownership, control, or status of resources held by a user, including accounts, properties, rights, and digital or physical items of value.

The term “document template” refers to a predefined document structure that includes placeholder fields or segments to be filled with user data, and that serves as a base for generating a document.

The term “document candidate information” refers to information representing a partially completed document obtained by assigning user data, including family structure information, wish information, and asset-related information, to corresponding fields in a document template.

The term “end-of-life record document” refers to a document that records a user's preferences, messages, or instructions relating to events or arrangements near or after the end of the user's life, without necessarily having binding legal effect.

The term “intention expression document” refers to a document in which a user expresses intentions, wishes, or guidance regarding personal, familial, or asset-related matters, regardless of its legal form.

The term “draft text” refers to text generated as a preliminary version of content for a document, which can be further edited or approved by a user before finalization.

The term “editable format” refers to a representation of text or document content that allows modifications by a user or application, including insertion, deletion, or alteration of characters, sentences, or sections.

The term “asset management information” refers to integrated data used for tracking, organizing, and updating information about a user's assets over time, including balances, ownership status, classifications, and historical changes.

The term “digital assets” refers to electronically represented resources associated with a user, such as digital accounts, digital currencies, licenses, access rights, or content stored in information systems.

In one embodiment, a server cooperates with at least one terminal and at least one storage device to implement the claimed system. The server executes a program stored in a non-transitory computer-readable medium. The program is implemented, for example, in a high-level programming language and runs on a general-purpose processor or a combination of central processing units and accelerator units such as graphics processing units. The server communicates with the terminal via a network using a communication protocol stack including at least a transport layer protocol and a secure application layer protocol.

The terminal includes a housing, a processor, a memory, a voice input device, a display device, and a communication interface. The voice input device includes at least one microphone, for example a micro-electro-mechanical system microphone, connected to an audio front-end circuit.

The terminal processor executes an operating system, such as a mobile operating system, and an application program that captures audio samples from the voice input device, buffers the audio samples in memory, and transmits the audio samples or converted text to the server. The display device is, for example, a liquid crystal display or an organic electroluminescent display driven by a display controller under control of the terminal processor.

The user operates the terminal by activating an application associated with the system, granting permission to access the microphone, network, and optionally health or location services. The user produces speech near the microphone. The voice input device converts the acoustic pressure of the speech into analog signals, which are digitized by an analog-to-digital converter and stored as a stream of digital samples in the terminal memory. The terminal normalizes, compresses, or segments this stream as required by a speech recognition service interface, and transmits it to the server or to a remote speech recognition engine.

The server converts the received digital audio into character information by applying a speech recognition technique. In one embodiment, the server uses an external speech recognition service that internally employs an acoustic model and a language model implemented by a deep neural network such as a recurrent neural network or a transformer network. In another embodiment, the server executes a local speech recognition module that performs feature extraction, such as Mel-frequency cepstral coefficient computation, followed by decoding using a hidden Markov model or a sequence-to-sequence model. The server outputs a sequence of characters corresponding to the recognized utterance of the user.

The server maintains user attribute information, such as preferred language, domain preferences, age range category, and previous interaction history, in at least one storage device. The server retrieves relevant user attribute information from the storage device in response to a recognition event and associates the attribute information with the corresponding character information. The server then analyzes the combined input information using a natural language processing pipeline.

In one embodiment, the server executes a natural language processing library that tokenizes the character information into tokens, assigns part-of-speech tags, and performs syntactic dependency parsing. The server further applies an entity recognizer to extract entities such as product categories, price constraints, health-related terms, or financial terms. The server uses a trained classifier, which can be a linear classifier, a gradient boosting model, or a small neural network, to determine a requested domain among at least a product information domain, a health support domain, an asset support domain, and a document creation domain. The classifier receives as input a feature vector generated from token n-grams, entity types, and user attribute information, and outputs a domain label with an associated confidence score.

The server generates processing request information based on the determined intention and the requested domain. For the product information domain, the server maps extracted entities to search condition information including category identifiers, maximum or minimum values for numeric attributes such as price, and quality-related constraints such as camera rating or battery capacity. For the health support domain, the server maps recognized symptoms, measurement terms, or time ranges to state evaluation information keys specifying which health indices to compute or retrieve. For the asset support domain, the server produces state evaluation information keys indicating which asset classes, time horizons, or risk measures to evaluate. For the document creation domain, the server generates a structured representation of family relations, wishes, and asset references.

The server stores product information, health information, and asset information in respective storage devices implemented as relational databases or non-relational data stores. Each storage device organizes data as structured records with indexed fields. For example, the product information storage device stores records including fields for item identifier, category identifier, price, specification fields, and location information codes corresponding to aisles and shelves in a physical facility. The health information storage device stores biometric and behavior measurements with timestamps. The asset information storage device stores balances, holdings, transaction history, and derived indicators.

The server searches the product information storage device using the search condition information. In one embodiment, the server translates the search condition information to a query expressed in a structured query language and submits the query to a database engine that uses index structures such as B-trees or inverted indices. The database engine returns matching records. The server then converts the records to structured data objects containing normalized attribute names and values. Similarly, the server retrieves relevant health or asset information using the state evaluation information keys, and aggregates this information into structured data.

For health information, the server may compute daily averages, rolling means, or standard deviations of measurements, and derive health indices such as activity scores or stability scores.

For asset information, the server may compute projected values using numeric methods such as compound interest formulas or Monte Carlo sampling, depending on configuration.

The server generates a prompt sentence for a generative AI model. The prompt sentence is text composed by concatenating template phrases, the recognized character information, and a textual representation of the structured data. The server may omit fields that are not relevant to the requested domain to reduce prompt size and improve computational efficiency. The server also includes instructions about desired style and length of the output. Because the server constructs the prompt sentence based on both character information and structured data, the generative AI model is not required to infer database content from user utterances alone, thereby reducing ambiguity and reducing unnecessary computation.

In one embodiment, for a product search scenario, the server generates a prompt sentence such as:
“Act as an in-store shopping assistant. The user said: ‘I am looking for a new smartphone under 500 dollars with a good camera.’ Available products: Product A, price 450, high camera quality, located at Aisle 3, Shelf B; Product B, price 399, medium camera quality, located at Aisle 3, Shelf C. Explain in simple English which product better matches the user's request, mention the prices and camera quality, and keep the answer under 120 words.”

In another embodiment, for an end-of-life document support scenario, the server generates a prompt sentence such as:
“You are assisting a user in drafting an ending note. The user has a spouse and two children. The user wishes to donate part of savings to charity and to ensure that the children can access a specific investment account. Draft a clear, polite summary paragraph in Japanese that the user can insert into an ending note to express these wishes to the family, avoiding specialized legal terminology and keeping the text concise.”

The server inputs the prompt sentence into a generative AI model. The generative AI model is implemented as a neural network having, for example, a transformer architecture with multiple self-attention layers, feed-forward sub-layers, and layer normalization. The model parameters, including weight matrices and bias terms, are stored in an accelerator memory and loaded into accelerator cores such as streaming multiprocessors during inference. The server passes the prompt sentence to a tokenizer that segments the text into subword tokens. The accelerator computes token embeddings, applies attention operations to compute context-sensitive representations, and outputs a probability distribution over possible next tokens at each generation step. The server decodes tokens using sampling or beam search with parameters such as temperature and top-k thresholds to obtain natural language text.

During training of the generative AI model, a training system minimizes a loss function such as cross-entropy between predicted and target tokens over large corpora, using gradient-based optimization such as stochastic gradient descent or adaptive methods. The training process includes backpropagation of errors through attention layers and feed-forward layers, and updates of parameters according to learning rate schedules. Data augmentation and regularization techniques such as dropout or label smoothing may be applied to improve generalization. The deployment model used by the server is a frozen version of the trained model.

The server receives the generated explanation information or recommendation information from the generative AI model and combines it with the previously generated structured data. The server formats the response information as a structured message that includes both human-readable text and machine-readable elements, such as item identifiers or coordinate codes, to enable the terminal to display responses and optionally support subsequent machine operations, such as plotting maps.

The server also extracts features from the voice signal to estimate an emotional state of the user. In one embodiment, the server computes prosodic features including pitch statistics, energy statistics, speaking rate, and spectral tilt over segments of the voice signal. These features are input to a classifier, which can be a convolutional neural network or a recurrent neural network trained on labeled emotional speech data. The classifier outputs an emotional state label and a confidence score. In another embodiment, the server fuses acoustic features with interaction features such as query repetition count or latency between responses to refine the emotional state estimation.

The server uses the estimated emotional state and the requested domain to dynamically adjust the contents of the prompt sentence and the expression style of the generated text. For example, if the emotional state is estimated as “confused,” the server modifies the prompt sentence to request a more step-wise, explanatory style and simpler vocabulary. If the emotional state is estimated as “frustrated,” the server may request shorter, more direct responses. This dynamic adjustment is implemented as rule-based transformations on the prompt generation templates, and leads to a different token distribution in the generative AI model, improving user comprehension and reducing the need for repeated queries. As a result, the server reduces the number of network round trips and inference calls required to satisfy a given information need.

The server acquires location information associated with product information when the system is used in a physical facility such as a retail store. The product information storage device stores, for each item, codes representing aisle identifiers, shelf identifiers, and optionally approximate coordinates within the facility. The server generates guidance information such as textual directions (“From the entrance, go straight to Aisle 3 and look at Shelf B on your right”) or step sequences. The server may also encode path information using a simple graph representation connecting aisles and nodes. By including such guidance information in the response information, the server enables the terminal to render a map or step-by-step instructions.

The terminal receives the response information from the server through the communication interface and parses it into display elements. The terminal uses a graphical user interface framework to display a list of products, health metrics, asset projections, or draft document sentences along with explanation information and recommendation information. For navigation, the terminal may overlay arrows or markers on a schematic floor plan. The terminal may use local sensors, such as short-range wireless transceivers or inertial measurement units, to estimate the user's approximate position within the facility and correlate it with location information in the response.

The user reviews the presented information and may provide follow-up voice input to refine a query or accept a recommendation. The terminal transmits follow-up character information and context identifiers to the server. The server references a dialogue history stored in a storage device. The dialogue history records previous character information, processing request information, structured data summaries, and sent responses. The server updates the prompt sentence and structured data by including relevant elements from the dialogue history, such as previously discussed product categories or health goals, so that the generative AI model can generate context-aware responses without reprocessing all earlier user inputs from scratch. This reuse of context reduces total computation and improves coherence.

In another embodiment, the server concentrates on health support. The terminal collects biometric information and behavior information from connected devices or platform services and periodically uploads them to the server. The server stores this information and computes health indices such as daily activity scores or stability metrics. The server constructs a prompt sentence that explicitly includes numerical values of the health indices and instructs the generative AI model to produce lifestyle guidance information constrained by those values. Because the server separates the numeric evaluation of indices from the generative description, the generative AI model does not need to perform arithmetic or statistical estimation and can focus on language generation. This division of roles improves calculation accuracy and reduces inference complexity.

In still another embodiment, the server supports asset condition analysis and document creation. The server receives asset-related information and computes projections using explicit algorithms such as discounted cash flow models. The server then generates a prompt sentence that summarizes computed projections and asks the generative AI model to produce a narrative explanation or a draft of an intention expression document. The generative AI model is thus driven by explicit numeric inputs and constraints. The server stores asset management information, including digital assets, in a unified data structure. The server can also feed parts of this information into a document template to produce document candidate information, which is then refined by the generative AI model based on prompt instructions.

By structuring all intermediate data as explicit structured data objects and by explicitly separating numeric computation from natural language generation, the server improves the technical behavior of the computer system. The server reduces duplicated parsing and arithmetic operations, lowers the computational load on the generative AI model, and allows the accelerator hardware to be used efficiently mainly for language generation tasks. The dynamic adjustment of prompt sentences based on emotional state and requested domain further improves efficiency by decreasing unnecessary iterations and misaligned responses. The unified pipeline, reusing the same modules for multiple domains, reduces memory footprint and context switching overhead, resulting in improved throughput and reduced latency.

In variations of the embodiment, the generative AI model may be replaced by a smaller domain-specific generative model, or a hybrid model that combines rule-based templates with neural network outputs. The emotion classifier may be implemented using different neural architectures or support vector machines. The databases may be replaced by different data storage technologies, such as key-value stores or columnar stores, as long as structured data is produced.

The system may be deployed in a cloud environment, an on-premises server, or an edge computing device, provided that the server executes the described program and cooperates with a terminal and storage devices. Through these embodiments and variations, the system achieves a technical improvement in the manner in which computer hardware and software cooperate to process voice-driven, multi-domain interactions, yielding faster, more accurate, and resource-efficient operation than conventional architectures.

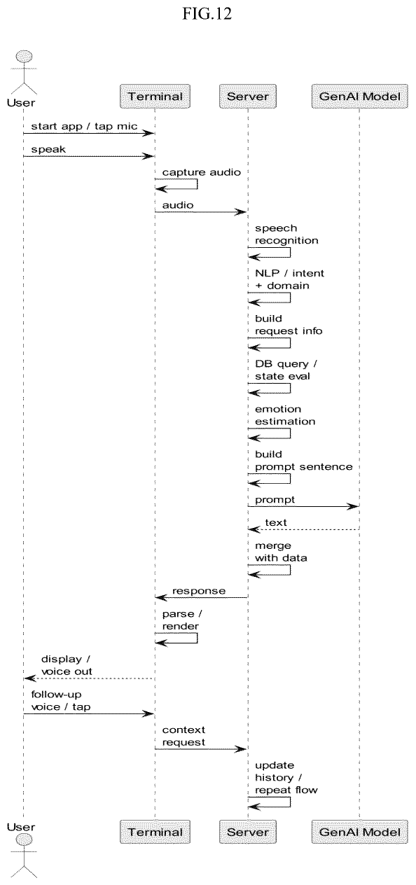

The following describes the processing flow using FIG. 12.

### Step 1:

The user activates an application on the terminal and initiates voice input.
Input: The user's spoken utterance and user actions such as tapping a voice button.
Output: A stream of raw audio samples stored in the terminal memory.

The terminal uses a voice input device to capture the user's speech, converts the analog signal into digital audio samples via an analog-to-digital converter, and stores the samples in a buffer. The terminal tags the buffer with metadata such as timestamp and session identifier.

### Step 2:

The terminal preprocesses the audio and sends it for speech recognition.
Input: Buffered raw audio samples and session metadata.
Output: A recognition request containing normalized audio data transmitted to a speech recognition engine.

The terminal applies data processing such as noise reduction, normalization, and optional compression to the audio samples, segments the samples into frames, and packages the processed audio into a request message. The terminal transmits this message via a network interface using a secure protocol.

### Step 3:

The server converts the audio into character information using a speech recognition technique.
Input: A recognition request containing processed audio data.
Output: Character information representing the recognized utterance.

The server performs feature extraction, such as computing spectral features and cepstral coefficients from the audio, and applies a decoding algorithm based on an acoustic model and a language model. The server executes probabilistic calculations to map feature sequences to a most likely sequence of characters and outputs text in a natural language.

### Step 4:

The server associates user attribute information with the recognized text.
Input: Character information and a user identifier derived from session metadata.
Output: Combined input information containing character information and user attribute information.

The server retrieves user attribute records from a storage device by performing a key-based lookup using the user identifier. The server merges fields such as preferred language, domain preferences, and past interaction indicators into a single data structure together with the character information.

### Step 5:

The server performs natural language analysis and domain classification.
Input: Combined input information including character information and user attribute information.
Output: A domain label and an intent representation.

The server tokenizes the character information into tokens, assigns part-of-speech tags, and applies an entity recognizer to extract entities such as product categories or health terms. The server generates a feature vector from token n-grams, entity types, and user attributes, and applies a trained classifier. The classifier computes scores for candidate domains and outputs a requested domain label and an intent label with a confidence score.

### Step 6:

The server generates processing request information.
Input: The domain label, intent label, extracted entities, and user attribute information.
Output: Processing request information that includes search condition information or state evaluation information.

The server applies rule-based mapping and parameter extraction. For example, if the domain label is product information, the server converts entities such as “smartphone” and “under 500 dollars” into category identifiers and numeric thresholds. For a health domain, the server converts recognized terms into codes for health indices. The server stores these mapped values in a structured object defining which data must be retrieved or computed.

### Step 7:

The server searches data storage devices and generates structured data.
Input: Processing request information containing search condition information or state evaluation information.
Output: Structured data representing retrieved or computed information.

The server translates search conditions into database queries and submits them to product, health, or asset information storage devices. The storage devices execute index lookups and filtering operations and return matching records. The server aggregates and normalizes the records into a uniform structured format. If state evaluation is required, the server performs calculations such as averaging measurements or projecting asset values and inserts the results into the structured data.

### Step 8:

The server extracts features from the voice signal and estimates an emotional state.
Input: Voice signal data or derived acoustic features and session metadata.
Output: An emotional state label and a confidence value.

The server computes prosodic and spectral features from the audio, including pitch statistics, energy, and temporal patterns. The server inputs these features into an emotion classification model, calculates activation values for each emotion category, and selects the category with the highest probability. The server outputs an emotional state label such as “calm” or “confused” and stores it in the session context.

### Step 9:

The server constructs a prompt sentence for a generative AI model.
Input: Character information, structured data, intent and domain information, and emotional state.
Output: A prompt sentence formatted as text for the generative AI model.

The server selects a template according to the requested domain and emotional state and fills placeholder fields with recognized text and structured data values. The server may add explicit instructions regarding tone, level of detail, and target length. The server concatenates these components into a continuous text string, forming a prompt sentence optimized for the generative AI model.

### Step 10:

The server invokes the generative AI model with the prompt sentence.
Input: The prompt sentence and generation parameters such as maximum length and sampling configuration.
Output: Explanation information and/or recommendation information in natural language form.

The server tokenizes the prompt sentence into subword tokens and sends them to a model-serving component. The generative AI model computes internal representations using a multi-layer neural architecture, applying matrix multiplications and attention operations. The server decodes the resulting token probabilities into a sequence of output tokens and converts them back to text, which constitutes generated explanation information or recommendation information.

### Step 11:

The server integrates generated text with structured data to form response information.
Input: Explanation information or recommendation information and structured data.
Output: Response information ready for transmission to the terminal.

The server combines human-readable text with machine-readable elements such as identifiers and location codes by embedding them in a response object. The server may append navigation descriptions based on location data or attach formatted numeric metrics. The server serializes this composite structure into a message format suitable for network transmission.

### Step 12:

The server adds navigation guidance when the requested domain involves product information in a physical facility.
Input: Structured product data including location identifiers and facility layout data from a storage device.
Output: Enhanced response information containing navigation guidance.

The server performs a lookup of aisle and shelf descriptors using location identifiers. If available, the server computes a path through a facility graph from a default entrance node to a target node.

The server translates this path into step-by-step textual instructions and appends these instructions to the response information.

### Step 13:

The server transmits the response information to the terminal.
Input: Response information object and addressing information for the terminal.
Output: A response message delivered over the network to the terminal.

The server encapsulates the response information in a network message, sets headers such as content type and session identifier, and sends the message using a secure communication protocol. The server may also log the response information along with request details into a dialogue history storage for later use.

### Step 14:

The terminal receives and parses the response information.
Input: The response message from the server.
Output: Parsed display data for the user interface and optional navigation data.

The terminal's communication interface receives the message and passes it to an application layer. The terminal parses the message into text segments, item lists, metrics, and navigation instructions. The terminal maps these elements to user interface components, such as list views and text fields, and stores any navigation-related data for rendering on a map.

### Step 15:

The terminal presents the response information to the user.
Input: Parsed display data and navigation data.
Output: Visual and/or audio output on the terminal.

The terminal renders text explanations, recommendations, and lists of items on the display, arranging them according to a predefined layout. If navigation guidance is included, the terminal displays arrows, route lines, or step instructions. The terminal may also convert text into speech using a text-to-speech engine and output audio through a speaker.

### Step 16:

The user reviews the output and optionally provides follow-up input.
Input: Visual or audio output and interactive UI controls on the terminal.
Output: New user commands or acceptance actions.

The user reads or listens to the information, scrolls through lists, or follows navigation directions.

The user may decide to refine the request, filter results, or confirm a recommendation, and operates controls such as buttons or voice input activation to send new input to the terminal.

### Step 17:

The terminal sends context-aware follow-up information to the server.
Input: New user input, current session context, and selected items or options.
Output: A context-enriched request message to the server.

The terminal packages the latest character information or voice data together with identifiers of previously selected items, last domain label, or navigation state. The terminal transmits this context-enriched message to the server so that the server can reuse dialogue history instead of restarting the processing from an empty context.

### Step 18:

The server updates the dialogue history and reuses prior structured data.
Input: Context-enriched request message and stored dialogue history records.
Output: Updated dialogue history and refined processing request information.

The server retrieves previous turns of the conversation from a storage device and merges the new input with earlier structured data. The server updates fields such as active filters or long-term goals. The server generates refined processing request information by adjusting search conditions or state evaluation parameters and repeats the analysis and prompt construction using the updated context.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional voice-based information management systems suffer from several technical limitations when transforming unstructured user speech into structured, shareable electronic documents. Typical systems merely transcribe audio into text and execute fixed command sets, without dynamically constructing machine-oriented prompts suitable for interaction with a generative AI model. As a result, the system cannot consistently generate high-quality, contextually appropriate documents such as health reports, asset summaries, and will drafts from heterogeneous and noisy user input.

Furthermore, known systems generally treat speech recognition, document generation, and cloud storage as disjoint functions. They do not define a coordinated processing pipeline in which (i) raw audio is converted into normalized text, (ii) a function-specific prompt sentence is algorithmically constructed for a generative AI model, (iii) the generated document is automatically formatted into a predetermined electronic document format, and (iv) the resulting file is uploaded to an external storage service with automatically generated share links. This lack of integration leads to increased latency, inconsistent document structure, and a heavy manual burden on the user to review, format, store, and distribute the generated content.

In addition, existing systems typically do not exploit acoustic features of the audio signal to estimate the emotional state of the user and feed that estimation back into the prompt construction process. Without emotion-aware prompt adaptation, the system cannot adjust the instruction content, writing style, or output format of generative AI outputs in a way that is responsive to user state or user attributes. This often yields documents that are technically correct but inappropriate in tone, level of detail, or complexity for the current user context.

Moreover, conventional data management solutions handle health-related data and asset-related data as simple time-series or record sets and require separate, manual analysis to obtain aggregated or trend-level insights. They do not provide a mechanism by which a processor aggregates such data, derives trend indices or valuation results, and then transforms these computational results into an information-provision prompt sentence for a generative AI model. Consequently, the system cannot automatically produce explanatory or summary documents that accurately reflect computed trends and valuations while remaining readable and understandable to non-expert users.

Therefore, there is a need for an improved computer-implemented system and processing architecture that: (i) tightly integrates speech acquisition, speech recognition, prompt construction, generative AI document generation, document post-processing, external storage, and link sharing; (ii) uses acoustic feature extraction and emotion estimation to dynamically adjust prompts and outputs; and (iii) programmatically aggregates health-related and asset-related data and converts those aggregation results into machine-oriented prompt sentences for generation of explanatory and summary documents. Such a system should reduce user interaction complexity, improve consistency and relevance of generated documents, and enhance overall performance and reliability of the underlying computer technology used for multimodal document generation and sharing.

The specific processing by the specific processing unit 290 of the data processing device 12 in Example 2 is realized by the following means.

The present invention provides a server comprising a processor configured to acquire audio data representing a user's voice via an acoustic input/output device, convert the acquired audio data into character information using a speech recognition algorithm, construct a prompt sentence serving as input to a generative AI model based on the converted character information and template information corresponding to a function type, input the constructed prompt sentence into the generative AI model to cause the generative AI model to generate document data including at least one of a report document, a record document, and an instruction document, perform formatting processing and additional-information attaching processing on the generated document data to generate file data in a predetermined electronic document format, upload the file data to an external storage service to obtain shared link information for accessing the file data, and transmit the shared link information to a user terminal for distribution to other parties, the processor further being configured to reconstruct, based on additional user input, a prompt sentence instructing modification or addition with respect to existing document data and regenerate updated document data by using the generative AI model, to extract time-domain and frequency-domain feature quantities from the audio data and estimate an emotional state of the user using an emotion estimation model, to dynamically adjust at least one of instruction content, writing style, and output format of the prompt sentence based on the estimated emotional state and user attribute information, and to perform aggregation processing on stored health-related data or asset-related data to derive aggregation results, generate an information-provision prompt sentence based on the aggregation results, and cause the generative AI model to generate at least one of an explanatory document and a summary document. This enables an integrated, computer-implemented pipeline that converts raw voice input into emotion-aware, function-specific prompts, leverages a generative AI model to produce structured electronic documents, automatically stores those documents in an external storage service with machine-generated share links, and programmatically generates explanatory and summary content from aggregated data, thereby improving the technical operation, efficiency, and usability of systems for multimodal document generation and sharing.

The term “processor” refers to a hardware-based or virtual computing element, such as a central processing unit, microcontroller, or processing core, that is configured to execute machine-readable instructions to perform the functions described herein.

The term “audio data” refers to digital data representing sound signals, including but not limited to sampled waveforms of a user's voice acquired via an acoustic input/output device.

The term “acoustic input/output device” refers to a hardware component, such as a microphone, speaker, or combination thereof, that is configured to capture or output sound signals for interaction with a user.

The term “character information” refers to digital text data obtained by converting audio data or other input into a sequence of characters, symbols, or tokens interpretable by a computer.

The term “speech recognition algorithm” refers to a software-implemented procedure or model that processes audio data and outputs a transcription of the spoken content as character information.

The term “template information” refers to predefined data structures or text patterns that specify a format, style, or content skeleton used when constructing a prompt sentence for a generative AI model.

The term “function type” refers to a classification or category indicating a target processing objective, such as generation of a health report, an asset summary, or a will-related document, which determines how a prompt sentence is constructed and how output is post-processed.

The term “prompt sentence” refers to a machine-readable text instruction or set of instructions, including context, constraints, and user-provided content, that is supplied as input to a generative AI model to control the model's output.

The term “generative AI model” refers to a trained computational model, such as a large-scale language model, that generates output data including text in response to input data such as a prompt sentence.

The term “document data” refers to structured or semi-structured text content generated by the generative AI model, including but not limited to report documents, record documents, and instruction documents.

The term “report document” refers to document data that presents analytical, descriptive, or summary information regarding a subject, such as a health condition or an asset status.

The term “record document” refers to document data that primarily serves to log, archive, or formally record information such as events, measurements, or user statements.

The term “instruction document” refers to document data that expresses commands, wishes, guidelines, or procedural steps, such as directions for asset distribution or health-related actions.

The term “formatting processing” refers to a sequence of operations that organize document data into a structured layout, including paragraphs, headings, lists, and page breaks, according to predetermined formatting rules.

The term “additional-information attaching processing” refers to operations that add metadata or auxiliary content to document data, such as timestamps, user identifiers, document titles, or annotations.

The term “file data” refers to a digital data structure in a predetermined electronic document format, generated from document data and suitable for storage, transmission, and display by computing devices.

The term “predetermined electronic document format” refers to a standardized file format, such as a portable document format or a word processing document format, defined in advance for representing electronic documents.

The term “external storage service” refers to a remote data storage system accessible via a communication network, such as a cloud-based object store or file hosting service, used to store and retrieve file data.

The term “shared link information” refers to data representing a network-accessible reference, such as a uniform resource locator or token, that permits controlled access to file data stored in an external storage service.

The term “user terminal” refers to an information processing device operated by a user, such as a mobile terminal, a tablet terminal, or a personal computer, configured to transmit and receive data to and from the server.

The term “additional input” refers to further user-provided data, including audio data or character information supplied after an initial document has been generated, which indicates modifications, corrections, or additions to existing document data.

The term “existing document data” refers to previously generated document data that is stored and subject to modification, augmentation, or replacement based on additional input.

The term “time-domain feature quantities” refers to numerical values derived from audio data in the time domain, such as energy, zero-crossing rate, or short-time amplitude statistics, used for subsequent analysis or classification.

The term “frequency-domain feature quantities” refers to numerical values derived from audio data in the frequency domain, such as spectral centroid, spectral bandwidth, or formant frequencies, used for subsequent analysis or classification.

The term “emotion estimation model” refers to a computational model that processes feature quantities derived from audio data and outputs an estimated emotional state of the user, such as calm, stressed, or sad.

The term “emotional state” refers to an inferred psychological or affective condition of a user, represented in discrete categories or continuous scales, estimated from input data such as audio signals.

The term “user attribute information” refers to data describing characteristics of a user, such as age group, language preference, expertise level, or profile settings, used to customize system behavior.

The term “instruction content” refers to the substantive commands, requests, or constraints expressed in a prompt sentence that direct the generative AI model regarding what to generate.

The term “writing style” refers to stylistic aspects of generated text, including formality level, tone, complexity, and narrative structure, which can be controlled through prompts.

The term “output format” refers to structural and presentational properties of generated text, such as sectioning, bullet-point use, length constraints, and inclusion of summaries, as specified or influenced by the prompt sentence.

The term “health-related data” refers to data describing a user's physical or mental condition, measurements, or behaviors, including but not limited to vital signs, test results, lifestyle logs, and symptom descriptions.

The term “asset-related data” refers to data describing a user's property or resources, including but not limited to financial holdings, real estate, digital assets, and valuation figures.

The term “aggregation processing” refers to computational operations that combine multiple data items, such as calculating sums, averages, counts, trends, or distributions over health-related data or asset-related data.

The term “aggregation result” refers to output data produced by aggregation processing, including numerical indicators, trend measures, and grouped or summarized datasets.

The term “information-provision prompt sentence” refers to a prompt sentence constructed to instruct the generative AI model to generate an explanatory or summary document based on aggregation results or other processed data.

The term “explanatory document” refers to document data that interprets, explains, or contextualizes underlying data or computation results in natural language suitable for human understanding.

The term “summary document” refers to document data that condenses underlying data or computation results into a concise representation highlighting key points, patterns, or conclusions.

The term “biological information” refers to health-related data that characterizes physiological conditions of a user, such as blood pressure, heart rate, body weight, or similar biometric parameters.

The term “life-log information” refers to time-series data describing daily activities, behaviors, or lifestyle factors of a user, such as exercise records, sleep duration, or dietary logs.

The term “trend index” refers to a computed indicator that expresses a tendency or temporal evolution of health-related data or life-log information, such as an increasing, decreasing, or stable pattern.

The term “health-condition explanation prompt sentence” refers to an information-provision prompt sentence specifically constructed to instruct the generative AI model to generate a document describing or explaining a user's health condition.

The term “health-condition report document” refers to a report document generated based on health-related data and trend indices, describing the user's health status, changes over time, and related observations.

The term “asset information” refers to asset-related data specifying the type, quantity, location, or value of a user's assets.

The term “inheritance-preference information” refers to character information representing a user's intended distribution or allocation of assets among beneficiaries or related parties.

The term “asset list data” refers to structured data listing a user's assets, categorized or grouped according to attributes such as asset type, ownership, or location.

The term “valuation data” refers to structured data representing monetary or other quantitative values assigned to assets based on evaluation or assessment.

The term “ending-related document” refers to document data associated with end-of-life planning, including but not limited to personal messages, instructions for care, or preferences regarding posthumous handling.

The term “will-related document” refers to document data that expresses testamentary intentions of a user, including allocation of assets and designation of beneficiaries.

The term “draft” refers to a preliminary version of an ending-related document or a will-related document generated by the generative AI model, intended for review, editing, or validation by the user or another party.

The term “specialist” refers to a person possessing expert knowledge in a relevant field, such as legal, medical, or financial expertise, who may review or utilize documents generated by the system.

The term “related person” refers to an individual or entity having a relationship or interest with respect to the user or the generated documents, such as a family member, caregiver, or designated representative.

In one embodiment, a server cooperates with at least one terminal used by a user to implement the system. The server includes a processor, a memory storing executable instructions and data structures, a network interface for communication over a data network, and an interface to an external storage service. The terminal includes a processor, a memory, an acoustic input/output device such as a microphone and a speaker, a display, and a communication module such as a wireless network interface.

The server stores and executes multiple software modules, including a speech-recognition interface module, a prompt-construction module, a generative AI inference module, a document post-processing module, a storage-and-link-management module, an emotion-estimation module, and a data-aggregation module. The server also stores relational or document-oriented database structures, including tables or collections for user profiles, raw input texts, prompt sentences, generated documents, file metadata, health-related data, and asset-related data.

The terminal executes an application that provides a graphical user interface to the user. The terminal uses an operating system-specific audio-recording library, such as a mobile operating system audio capture API, to acquire audio data from the microphone. The terminal uses a software development kit or network API to send the audio data and user commands to the server, to receive generated documents and share links from the server, and to display content on the display.

The server receives audio data and converts the audio data into character information. The server uses a speech recognition algorithm that may be implemented as a deep neural network acoustic model combined with a language model. In one embodiment, the server uses an encoder-decoder architecture with a recurrent neural network or transformer-based encoder that converts a sequence of audio feature vectors (for example, Mel-frequency cepstral coefficients or log-Mel filterbank energies) into a sequence of probability distributions over subword units. The server uses a beam search decoding process with a language model to produce a stable transcription string as character information. The server stores the resulting text in a user_inputs table, with fields including user_id, input_type, raw_text, timestamp, and language_code.

The server constructs a prompt sentence for a generative AI model based on the character information and a function type. The server stores, in memory or in a configuration database, template strings associated with each function type. For example, for a health-report function type, the server stores a template such as:
“You are a medical documentation assistant. Based on the following patient statement, create a clear and structured health report in English. Statement: [USER_TEXT].”

For an asset-management and will-drafting function type, the server stores a template such as:
“You are a legal document drafting assistant. Based on the following asset information and testamentary wishes, draft a formal will in clear legal language. Assets and wishes: [USER_TEXT]. Please include appropriate clauses about asset distribution, beneficiaries, and general provisions.”

The server replaces a placeholder token [USER_TEXT] with the actual user text obtained from the speech recognition algorithm and thereby generates a concrete prompt sentence. The server may also include additional context fields, such as “User age group: [AGE_GROUP]” or
“Preferred tone: [TONE],” into the prompt sentence. The server records each constructed prompt sentence in a prompts table with a foreign key referencing the corresponding user_inputs record.

The server uses a generative AI model to generate document data from the prompt sentence. In one embodiment, the server hosts or accesses a transformer-based language model trained on a large corpus of text. The generative AI model includes multiple self-attention layers, feed-forward sublayers, layer normalization, and learned positional encodings. The server supplies the prompt sentence to the model along with parameters such as a maximum token length, a temperature parameter controlling sampling randomness, and a top-k or nucleus sampling threshold. The generative AI model processes the input sequence token by token, using multi-head self-attention to compute context-aware representations, and produces output tokens that the server concatenates into a generated document text.

The server achieves a technical improvement in text generation accuracy and processing efficiency by constructing function-specific prompt sentences that encode both user intent and structured instructions. The server uses these structured prompt sentences to bias the generative AI model toward outputs that match predefined document patterns, which reduces the need for extensive post-editing and increases the consistency of the generated documents across different sessions and users. This structured prompting differs from manual human drafting or naive text generation in that the prompts are programmatically synthesized from data structures and templates, and their structure is automatically adapted based on function type and emotion estimation results.

The server performs post-processing on the generated document text. The server uses a text-processing library to normalize whitespace, correct heading capitalization, and insert section labels such as “Summary,” “Details,” and “Recommendations.” The server may insert metadata fields at the beginning or end of the document, such as user identifier, generation timestamp, function type, and model version. The server then converts the processed text into file data in a predetermined electronic document format. For example, the server uses a library to create a portable document format file with page headers, footers, and page numbers, or a word-processing format file with defined styles for headings and paragraphs. The server stores a record in a generated_documents table that references the input, the prompt, and the file location.

The server uploads the file data to an external storage service. The server uses a network client library or a storage service software development kit to perform an authenticated upload of the file to a cloud storage bucket or folder. The server sets access-control properties, such as a setting that the file is private by default. The server then requests the storage service to create shared link information, such as a uniquely generated uniform resource locator and an access token, with specified permissions (for example, read-only access for any holder of the link). The server stores the shared link information in a files table with fields including file_id, user_id, document_id, storage_location, and share_url.

The terminal receives the shared link information from the server and displays it to the user. The terminal may present the link as a button labeled “Open report” or “Share with family.” The user instructs the terminal to share the link through an email client or messaging application. The terminal uses the operating system's share interface to propagate the shared link information to other installed applications. This cooperation between server and terminal yields a concrete technical effect, namely, automatic generation and distribution of formatted documents through a network, without requiring the user to manually copy, store, and attach file data.

The server estimates an emotional state of the user from the audio data. Before transcription or in parallel with transcription, the server computes audio feature vectors. The server computes time-domain features such as short-time energy, zero-crossing rate, and amplitude variance over frames of a fixed window size. The server computes frequency-domain features such as spectral centroid, spectral roll-off, spectral flux, and Mel-frequency cepstral coefficients over the same frames. The server concatenates these features into a feature vector sequence. The server inputs this sequence into an emotion estimation model, which may be implemented as a convolutional neural network followed by a recurrent neural network layer or a transformer encoder that aggregates temporal information. The emotion estimation model is trained using supervised learning on labeled emotional speech data, using a loss function such as cross-entropy between predicted emotion probabilities and ground-truth labels. The server updates model weights during training by stochastic gradient descent or an adaptive optimization algorithm until a convergence criterion is met.

The server uses the estimated emotional state, such as “calm,” “stressed,” or “sad,” together with user attribute information, to dynamically adjust the prompt sentence. For example, if the emotion estimation model outputs a high probability for a stressed state, the server may modify the prompt sentence to request more concise and reassuring text. An example of an adjusted prompt sentence for a health report is:
“You are a medical documentation assistant. The patient is currently under stress. Based on the following patient statement, create a clear, concise, and reassuring health report in simple English. Avoid technical jargon. Statement: [USER_TEXT].”

By programmatically modifying the prompt sentence based on measured acoustic features, the server implements a specific non-human rule set to adapt the generative AI output. This method differs from human post-editing, because the adaptation is performed automatically before generation based on numerical feature analysis, and the modification rules are defined in configuration data and executed as part of the server's control logic. This yields a technical effect of reducing the amount of output that is unsuitable in tone and lowering the number of corrective user interactions required, thereby improving throughput and reducing network and computation overhead for repeated generations.

The server aggregates health-related data or asset-related data and constructs information-provision prompt sentences. The server stores health-related data in a structured form, such as a relational database table with columns for measurement type, value, timestamp, and unit. The server retrieves data over a specified time window and computes aggregation results such as average, minimum, maximum, and variance values, as well as trend indices computed by fitting a simple regression model or by computing the sign and magnitude of differences over time. For example, the server calculates a blood pressure trend index as the slope of systolic and diastolic values over a 30-day period.

The server constructs a health-condition explanation prompt sentence that encodes these computed values explicitly. An example is:
“You are a medical report generator. Here are three months of blood pressure and weight records for a patient, including computed averages and trends. Average systolic blood pressure: 122 mmHg, trend: slightly increasing. Average diastolic blood pressure: 78 mmHg, trend: stable. Average weight: 68 kg, trend: decreasing. Create a three-page English health status report summarizing trends, highlighting any risks, and providing clear explanations suitable for a general practitioner.”

The server supplies this prompt sentence to the generative AI model. By feeding the numerical aggregation results as structured text into the model, the server ensures that the model output directly reflects the computations performed by the server. This partition of responsibilities, where the server performs numerical aggregation and the generative AI model performs language realization, improves the accuracy and reproducibility of the generated reports compared with systems that attempt to infer trends purely from narrative descriptions. The technical effect is improved reliability and consistency of trend-based explanations and reduced computational load on the generative AI model, because the model does not need to infer statistical properties from raw time-series data.

The server similarly aggregates asset-related data. The server stores asset information with fields such as asset_category, asset_name, quantity, unit_value, and total_value. The server computes valuation data by multiplying quantity by unit_value or by applying a valuation function that may access external market data. The server generates asset list data by grouping assets by category and sorting them by value. The server constructs a prompt sentence such as:
“You are a legal document drafting assistant. Based on the following classified asset list and valuation data, and the user's inheritance preferences, draft a formal will in clear legal language.
Asset list: [ASSET_LIST_DATA]. Inheritance preferences:
[INHERITANCE_PREFERENCES]. Include clauses about asset distribution, beneficiaries, and general provisions.”

The server passes this prompt sentence to the generative AI model. Because the asset list data and valuation data are precomputed and structured, the generative AI model can focus on drafting correct legal language rather than computing totals or valuations. This division of tasks reduces the risk of numerical errors in the generated documents and improves the determinism of outputs when the underlying data remains unchanged.

The server improves computer technology by defining an integrated data flow and specialized data structures for prompt construction, emotion estimation, and aggregation-based explanation. In conventional systems, audio, text, and documents might be handled in separate modules with weak coupling. In this system, the server enforces a specific sequence of conversions and maintains referential integrity between audio records, transcriptions, prompt sentences, and generated documents using unique identifiers in database tables. This architecture allows efficient indexing, caching of generation results, and incremental updates when the user provides additional input. When the user requests a revision, the server retrieves the original prompt, the original generated text, and the additional input, and reconstructs a new prompt sentence that explicitly refers to the previous version, for example:
“You previously drafted the following will: [ORIGINAL_DOCUMENT]. The user now requests the following changes: [USER_ADDITIONAL_INPUT]. Revise the will accordingly, preserving all other provisions.”

This re-use of stored artifacts yields a reduction in network load and processing time, because the system can limit re-generation to only the affected documents and use prior outputs as context.

The server can be implemented with different variations of hardware and software components. In one embodiment, the server uses a central processing unit cluster and a graphics processing unit or specialized accelerator to execute the generative AI model and the emotion estimation model. In another embodiment, the server accesses a remote inference service that hosts the generative AI model, while locally executing only the speech recognition and emotion estimation algorithms. In yet another embodiment, part of the speech processing or emotion estimation is offloaded to the terminal, which contains a dedicated digital signal processor. These variations allow scaling and optimization of computational resources while preserving the same logical flow and technical advantages of the described architecture.

The server trains the generative AI model and the emotion estimation model using standard machine learning techniques but with specific configurations oriented toward the described tasks. The server uses mini-batch training, backpropagation, and an error function such as cross-entropy or mean-squared error to update weights. The server applies data augmentation methods in the audio domain, such as adding noise, shifting pitch, or varying speed, to increase robustness of emotion estimation. The server also may use prompt-tuning or fine-tuning techniques on the generative AI model with domain-specific text corpora to enhance the accuracy of health-related and legal-related documents. These training choices contribute to a technical effect of improved recognition and generation accuracy, reduced failure rates, and stable performance across different acoustic conditions and user populations.

The terminal cooperates with the server to reduce communication load and latency. The terminal may perform local buffering of audio data and compression before upload, and may implement a partial on-device speech recognition model to provide immediate feedback to the user while the server performs high-accuracy recognition and generation. The terminal can cache recently received documents and share links to avoid redundant requests to the server. This coordinated design contributes to improved response time and reduced network traffic.

By implementing these concrete data structures, algorithms, and module interactions, the server and the terminal together provide a system that goes beyond a mere automation of human document drafting. The system introduces specific computational rules for constructing prompt sentences, estimating emotional states from numerical feature quantities, and translating numerical aggregations into explanatory text via a generative AI model. These rules and data flows yield measurable improvements in processing speed, generation accuracy, document consistency, and resource utilization, thereby constituting an improvement in the functioning of the computer-based system itself.

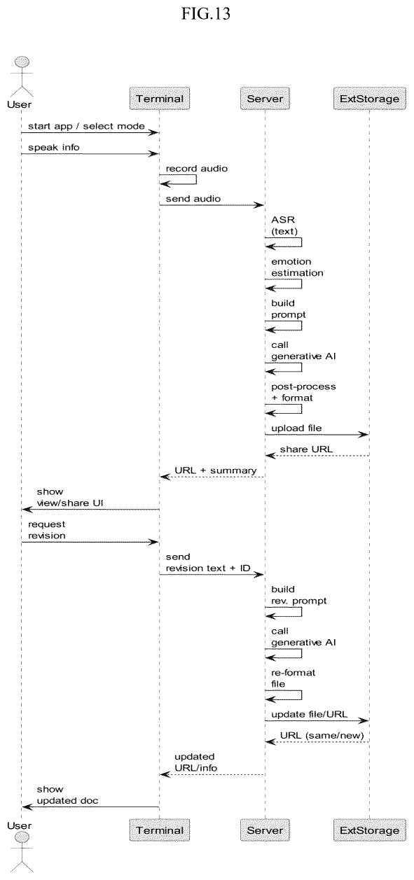

The following describes the processing flow using FIG. 13.

### Step 1:

The user operates the terminal to start an application and select a function type (for example, health report, asset management, or will drafting). The terminal receives user input events (touch, click, or voice command) as input and outputs a function-type identifier and a session context.

The terminal stores the selected function type in local memory and displays an appropriate input screen indicating that audio input will be used.

### Step 2:

The user speaks into the microphone of the terminal to input information such as health data, asset information, or testamentary wishes. The terminal receives analog voice signals as input and outputs digital audio data. The terminal uses an audio capture API to sample the analog signal at a defined sampling rate (for example, 16 kHz) and quantization (for example, 16-bit PCM), segments the audio into frames, and writes the audio frames into a buffered audio file in local storage, while updating a recording indicator on the display.

### Step 3:

The terminal sends the recorded audio data and metadata to the server. The terminal takes as input the buffered audio file and associated metadata such as user identifier, function type, and timestamp, and outputs an HTTP request message. The terminal encapsulates the audio data in a multipart or binary payload, attaches headers including authentication information and content type, and transmits the message through a network interface to the server over a secure protocol.

### Step 4:

The server receives the audio data and stores it in a database or file storage. The server takes as input the network request containing audio data and metadata, and outputs a stored audio record and an internal audio identifier. The server parses the request, validates authentication tokens, writes the raw audio bytes to a storage location, stores a record in an audio_inputs table including user_id, function_type, file_path, and timestamp, and returns the generated audio identifier to the application logic.

### Step 5:

The server converts the audio data into character information using a speech recognition algorithm. The server takes as input the stored audio data and the audio identifier, and outputs a transcription string. The server computes audio features (such as Mel-frequency cepstral coefficients) from the audio frames, inputs the feature sequence into a trained neural speech recognition model, calculates probability distributions over subword units for each time step, performs beam search with a language model to select the best sequence of tokens, and concatenates the tokens into a text string representing the user's speech.

### Step 6:

The server stores the transcription as raw text linked to the user and the audio input. The server takes as input the transcription string and metadata including user_id and function_type, and outputs a new user_inputs record. The server inserts a row into a user_inputs table containing fields such as input_id, user_id, audio_id, raw_text, language_code, and creation_time, and returns the input_id for subsequent processing.

### Step 7:

The server extracts audio features and estimates the user's emotional state. The server takes as input the same audio data associated with the audio_id, and outputs an emotion label or probability vector. The server computes time-domain features (for example, frame-level energy, zero-crossing rate) and frequency-domain features (for example, spectral centroid, spectral roll-off, Mel-frequency cepstral coefficients), stacks them into a feature vector sequence, feeds the sequence into an emotion estimation model (such as a convolutional and recurrent neural network), calculates scores for multiple emotion categories using a softmax layer, and selects an emotion label such as “calm,” “stressed,” or “sad” based on maximum probability.

### Step 8:

The server constructs a prompt sentence for a generative AI model using the transcription, function type, emotion label, and user attributes. The server takes as input the raw_text, function_type, emotion label, and user profile data, and outputs a prompt sentence string. The server selects a template string corresponding to the function_type (for example, a health-report template or will-drafting template), replaces placeholders such as [USER_TEXT], [EMOTION_STATE], or [PREFERRED_TONE] with the actual values, concatenates additional instructions (for example, “use simple language” or “use formal legal language”), and generates a complete prompt sentence that explicitly describes the required output structure.

### Step 9:

The server stores the constructed prompt sentence in association with the input record. The server takes as input the prompt sentence and the input_id, and outputs a new prompts record. The server inserts a row into a prompts table containing fields such as prompt_id, input_id, function_type, prompt_text, emotion_label, and timestamp, thereby enabling traceability between the original audio, the transcription, and the prompt used for generation.

### Step 10:

The server invokes the generative AI model with the constructed prompt sentence to generate document data. The server takes as input the prompt sentence and model configuration parameters (such as maximum tokens and temperature), and outputs generated text content. The server sends the prompt sentence as input tokens to a transformer-based language model, computes self-attention and feed-forward transformations across multiple layers, iteratively predicts next tokens using sampling or greedy decoding, and composes the generated tokens into a document text representing a report, record, or instruction document.

### Step 11:

The server stores the generated document data in the database. The server takes as input the generated document text and identifiers such as prompt_id and input_id, and outputs a generated_documents record. The server writes a row including document_id, prompt_id, input_id, document_text, document_type, and generation_time to a generated_documents table, making the document retrievable for later revision or sharing.

### Step 12:

The server performs formatting and metadata attachment to convert the generated document text into file data. The server takes as input the document_text and document_id, and outputs a formatted electronic document file. The server uses a document generation library to parse the document_text, create sections and headings, apply a predefined style sheet, insert metadata such as author, creation date, user identifier, and document type, and render the content into a predetermined electronic document format such as a portable document format file or a word-processing file stored temporarily on disk.

### Step 13:

The server uploads the formatted file data to an external storage service and generates shared link information. The server takes as input the local file path and metadata including user_id and document_id, and outputs a storage location identifier and a shareable link. The server calls an external storage application programming interface to upload the file, receives an object identifier or file key, then requests the external storage service to create an access link with specified permissions, receives a uniform resource locator or token, and stores a record in a files table including file_id, document_id, storage_key, and share_url.

### Step 14:

The server transmits the shared link information and a summary of the document to the terminal. The server takes as input the share_url and document metadata, and outputs a response message to the terminal. The server composes a response object containing the document_id, document_type, a short text summary extracted from the beginning of the document_text or generated explicitly, and the share_url, and sends this object over the network to the terminal.

### Step 15:

The terminal receives the response and updates the user interface to show the newly generated document. The terminal takes as input the response message containing the share_url and summary, and outputs updated display content and interaction options. The terminal parses the response, stores the share_url and document_id in local storage, displays a notification such as “New report available,” presents a “View document” button that opens the share_url in a viewer, and provides a “Share” button that allows distribution of the link through communication applications.

### Step 16:

The user reviews the document and may request a revision by providing additional input. The user takes as input the displayed document contents and any perceived need for changes, and outputs additional instructions by typing or speaking, such as “Change the percentage for my spouse to 60 percent” or “Summarize only the blood pressure data.” The terminal captures this additional input as text or audio, displays the text for confirmation, and associates it with the original document_id.

### Step 17:

The terminal sends the additional input and the reference to the existing document to the server. The terminal takes as input the additional instruction text and the document_id, and outputs a structured revision request. The terminal includes in the request the user_id, document_id, original function_type, and the new instruction text, and transmits the revision request to the server over the network.

### Step 18:

The server reconstructs a new prompt sentence for revision based on the original document and the additional input. The server takes as input the document_id and the additional instruction text, and outputs a revised prompt sentence. The server retrieves the original document_text and prompt_text from the database using document_id, composes a new prompt sentence that includes both the original content and explicit instructions for modification (for example, “You previously drafted the following document: [ORIGINAL_DOCUMENT]. The user now requests: [ADDITIONAL_INSTRUCTION]. Revise the document accordingly.”), and stores this revised prompt in the prompts table with a link to the same input record.

### Step 19:

The server invokes the generative AI model again with the revised prompt sentence to generate updated document data. The server takes as input the revised prompt sentence and model parameters, and outputs a new document_text replacing or augmenting the prior version. The server performs the same sequence of generative computations as in the initial generation, but with the revised prompt, then updates the corresponding generated_documents record or creates a new version record indicating that this is a revision.

### Step 20:

The server regenerates the formatted file and updates or creates shared link information as appropriate. The server takes as input the updated document_text and the existing file metadata, and outputs an updated file and share_url. The server re-renders the electronic document file to reflect the new content, either overwrites the file at the external storage service or uploads a new version, and if necessary, generates a new share_url or preserves the existing one, then notifies the terminal of the updated state.

### Step 21:

The server periodically aggregates stored health-related data or asset-related data to prepare information-provision documents. The server takes as input collections of records from health-related or asset-related data tables, and outputs aggregation results and related documents. The server executes database queries to select records for a specified period, performs arithmetic operations such as averaging, summing, variance calculation, and trend detection, then converts these numerical results into structured text fragments that are embedded into an information-provision prompt sentence, which is then supplied to the generative AI model to produce explanatory or summary document_text that is stored, formatted, and shared in the same manner as other documents.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional voice-driven assistance systems typically implement a linear pipeline in which user speech is converted to text and then mapped, through relatively static rules, to individual application functions. Such systems exhibit several technical limitations.

First, existing architectures generally treat intent understanding, emotion recognition, and back-end service control as separate subsystems with weak coupling. The speech recognition component outputs text; an independent intent classifier selects an application; and a separate user-interface layer determines how responses are presented. This fragmented design leads to repeated parsing of the same data, redundant computation, and increased latency on constrained devices and networks. It also prevents the system from globally optimizing processing flows based on a unified view of user intent, emotional state, and historical context.

Second, conventional systems do not systematically integrate generative AI models into the control plane of the system. Generative models, if used at all, are often invoked as passive text generators, receiving ad-hoc prompts manually constructed at the application layer. Such ad-hoc prompting does not exploit structured metadata (user identifiers, timestamps, use-purpose tags, emotion labels, historical records), and therefore forces the surrounding software to perform substantial pre- and post-processing to correct, constrain, or reinterpret the AI output. This results in brittle behavior, unpredictable outputs, and additional error-handling code, thereby degrading the reliability and scalability of the overall computer system.

Third, known systems frequently process emotion signals only at the user-interface level, for example to change wording or show certain icons, without allowing the emotion analysis to influence security controls, data routing, or resource allocation. As a result, computing resources are not prioritized for urgent or high-risk situations, and the system cannot programmatically escalate to secure channels, emergency notification modules, or different back-end pipelines. This deficiency manifests as slow or inappropriate handling of emotionally critical interactions, despite the presence of sufficient hardware and network capacity.

Fourth, when sensitive information such as health records, financial records, or end-of-life documentation is generated through voice interaction, conventional systems usually handle encryption, external storage, and access control as independent layers. For example, an application may write plain text to storage and rely on an external security module to encrypt and manage access. This fragmented approach complicates the data path, introduces multiple transfer points for unencrypted data, and increases the risk of inconsistent access-right enforcement across services and clients. Additionally, existing systems rarely bind access-control policies to the same unified intent-and-emotion context that governs function execution, making it difficult to dynamically adjust sharing and notification behavior based on the content and emotional criticality of a given interaction.

Fifth, most known architectures lack a unified framework in which continuous sensor-derived data (e.g., physiological measurements, activity logs) and episodic voice-interaction data (speech, text, emotion) are co-processed and summarized into machine-usable indices. Health-related or behavioral data is often analyzed in isolation, with separate code paths and data stores, so that the system cannot efficiently compute indices once and then reuse them across multiple AI-driven functions. This redundancy leads to repeated scanning and aggregation of large data sets, wasting processor cycles and memory bandwidth and increasing response times for downstream services.

Accordingly, there is a need for an improved computer-implemented system in which a processor unifies (i) acquisition and encoding of user voice signals, (ii) speech-to-text conversion with structured metadata binding, (iii) multi-modal emotion analysis from text and acoustic features, (iv) structured prompt-sentence generation for a generative AI model using intent, emotion, and historical context, (v) derivation of concrete processing plans from the model's responses, and (vi) integrated encryption and access-right management for the resulting sensitive data. There is also a need for such a system to reuse computed health or behavior indices across multiple services, to condition emergency handling and presentation modalities on emotion analysis, and to thereby reduce redundant computation, lower latency, and increase reliability and security of the overall computer system.

The specific processing by the specific processing unit 290 of the data processing device 12 in Application Example 2 is realized by the following means.

The present invention provides a server comprising a processor configured to acquire a user voice signal from an input device and encode the user voice signal as time-series data; to convert the time-series data into character information by applying a speech recognition process and to store the character information in association with user identification information, time information, and usage-purpose information in a unified data structure; to extract acoustic feature values from at least one of the user voice signal and the time-series data; to generate an emotion analysis result by classifying an emotional state of the user based on the character information and the acoustic feature values and to store the emotion analysis result in association with the character information; to derive an intent determination result indicating an intent category from the character information; to construct, based on the intent determination result, the emotion analysis result, and attribute information or history information relating to the user, a structured prompt sentence to be input to a generative information processing model; to input the prompt sentence to the generative information processing model, obtain response information from the generative information processing model, analyze the response information, and identify a processing plan or guidance information relating to at least one of health information processing, asset information processing, life information processing, end-of-life record support, and store information support; to adjust content or a mode of presentation of information to the user based on the processing plan or the guidance information and the emotion analysis result and to execute, in accordance with predetermined conditions, at least one of an emergency contact process, a monitoring process, and a psychological burden-reduction process; to generate confidential data by subjecting privacy-sensitive information, which is generated based on the character information or the processing plan, to an encryption process, store the confidential data in an external storage device, and manage access-right information for the confidential data on a per-user-group basis; and to perform authentication and authorization for a reference request to the confidential data based on the access-right information and to provide the confidential data or information decrypted from the confidential data to a terminal device having authorization. This enables the computer system to treat intent analysis, emotion recognition, AI-driven planning, and secure data management as an integrated control flow, thereby reducing redundant computations across subsystems, lowering end-to-end latency for voice-driven services, dynamically prioritizing and routing processing based on emotional urgency, and strengthening confidentiality and consistency of access-right enforcement for sensitive information generated through the system.

The term “voice signal” refers to an analog or digital representation of sound produced by a user, including speech, that is suitable for acquisition by an input device and subsequent processing as time-series data.

The term “input device” refers to any hardware component configured to capture user speech, such as a microphone or an audio sensor, and to output corresponding electrical or digital signals to the processor.

The term “time-series data” refers to a sequence of numerical values representing a signal, such as audio, sampled at successive points in time at a defined sampling rate.

The term “speech recognition process” refers to a computational procedure that analyzes time-series audio data and converts the data into corresponding character information, such as text, using pattern recognition or machine learning techniques.

The term “character information” refers to digital data representing linguistic content, including letters, numbers, symbols, and words, obtained by converting a voice signal or other input into text form.

The term “user identification information” refers to data that uniquely or pseudo-uniquely identifies a user within a system, such as a user ID, account ID, or device-linked identifier.

The term “time information” refers to data indicating a temporal attribute of an event or record, such as a timestamp representing a date and time at which speech was acquired or processed.

The term “usage-purpose information” refers to data that specifies an intended category of processing or application context associated with an item of character information, such as health management, financial management, or store information support.

The term “acoustic feature values” refers to numerical descriptors extracted from a voice signal or time-series data, such as frequency components, energy, pitch, formants, or temporal dynamics, used for tasks including emotion classification.

The term “emotion analysis result” refers to data indicating a classified emotional state of a user, such as “anxious” or “calm,” and optionally including a confidence value, derived from analysis of character information and acoustic feature values.

The term “intent determination result” refers to data representing an inferred category of user intent, such as health inquiry, end-of-life documentation request, financial question, or store inventory query, derived from analysis of character information.

The term “intent category” refers to a predefined or dynamically defined class of processing or service corresponding to a type of user intention, used to route a request to an appropriate function.

The term “attribute information” refers to data describing characteristics of a user, such as demographic information, preference information, usage patterns, or device capabilities, that may influence system behavior.

The term “history information” refers to data representing prior interactions or records associated with a user, including past utterances, past emotion analysis results, or past service usage logs.

The term “prompt sentence” refers to a structured text input formulated for a generative information processing model, containing instructions, context, and user-related data to guide the model's generation of response information.

The term “generative information processing model” refers to a computational model, such as a generative AI model, configured to generate text or other data outputs in response to input prompts, based on learned statistical relationships.

The term “response information” refers to output data generated by the generative information processing model in response to a prompt sentence, including explanations, instructions, or plans for subsequent processing.

The term “processing plan” refers to data describing one or more actions, operations, or function calls to be executed by the system in order to fulfill a user's intent, as identified by analyzing response information.

The term “guidance information” refers to data representing advice, explanations, or procedural instructions that are intended to be presented to a user to support a task or decision.

The term “health information processing” refers to computational operations that store, analyze, or present data related to physical or mental health, such as physiological measurements or lifestyle metrics.

The term “asset information processing” refers to computational operations that store, analyze, or present data related to financial or property resources, such as income records, expenditures, or holdings.

The term “life information processing” refers to computational operations that handle general daily-life related data, including schedules, reminders, shopping information, or household management information.

The term “end-of-life record support” refers to computational operations that assist a user in creating, managing, or organizing documentation expressing wishes or instructions related to end-of-life matters, including wills and personal notes.

The term “store information support” refers to computational operations that acquire, search, or present information related to products, inventory, prices, or promotions in a commercial environment.

The term “mode of presentation” refers to a manner in which information is output to a user, including modality (such as audio or visual), formatting, level of detail, or tone.

The term “emergency contact process” refers to a sequence of computational operations that, upon detecting certain conditions, initiate communication to emergency contacts or services using messaging, calling, or other notification mechanisms.

The term “monitoring process” refers to continuous or periodic computational operations that observe user state, sensor data, or system interactions to detect changes or abnormal patterns.

The term “psychological burden-reduction process” refers to computational operations that, based on an emotion analysis result, provide content such as calming messages, relaxation guidance, or supportive feedback to alleviate user stress or anxiety.

The term “privacy-sensitive information” refers to data that, if improperly disclosed, could affect a user's privacy, including health data, financial data, identification data, or end-of-life documentation.

The term “encryption process” refers to a computational transformation that converts plain data into cipher data using an encryption algorithm and a cryptographic key so that the data cannot be understood without decryption.

The term “confidential data” refers to encrypted data produced by an encryption process from privacy-sensitive information and stored to prevent unauthorized access.

The term “external storage device” refers to any storage resource external to the processor's primary memory, such as a network-attached storage, remote database, or cloud storage service.

The term “access-right information” refers to data representing authorization policies or permissions that specify which entities or groups may access, decrypt, or operate on particular confidential data.

The term “reference request” refers to a request issued by a terminal or process to read, retrieve, or otherwise gain access to stored data, including confidential data.

The term “authentication” refers to a computational process that verifies an identity of a requesting entity based on credentials such as identifiers, tokens, or cryptographic proofs.

The term “authorization” refers to a computational process that determines whether an authenticated entity has permission, under access-right information, to perform a requested operation on particular data or resources.

The term “terminal device” refers to any user-operated computing apparatus, such as a smartphone, tablet, wearable device, or personal computer, that communicates with the server and presents information to the user.

The term “health-related data” refers to data measured or inferred about a user's physical or mental condition, such as heart rate, activity level, sleep pattern, or stress indicator.

The term “biological measurement device” refers to any sensor or apparatus configured to measure physiological parameters of a user, such as heart rate, blood pressure, or body temperature.

The term “behavior measurement device” refers to a sensor or apparatus configured to measure user behaviors or activities, such as movement, steps, or sleep duration.

The term “health-state index” refers to a computed metric or set of metrics summarizing a user's health status, derived from health-related data by statistical or trend-analysis processing.

The term “health-management information” refers to data including evaluations, alerts, or recommendations generated based on a health-state index and optionally on response information from a generative information processing model.

The term “intention content” refers to textual or structured data representing the substantive wishes, requests, or instructions expressed by a user, especially concerning end-of-life or testamentary matters.

The term “property information” refers to data describing assets or liabilities associated with a user, including financial accounts, physical properties, or digital assets.

The term “family-structure information” refers to data describing relationships among persons associated with a user, such as kinship, dependents, or designated beneficiaries.

The term “document structure” refers to a logical or syntactic organization of a document, such as sections, clauses, and fields, which can be populated with content to form an end-of-life record or testamentary record.

The term “auxiliary information” refers to supplementary data, such as examples, clarifications, or contextual notes, provided to assist a user in understanding or completing a task.

The term “end-of-life record” refers to a document or data set that expresses a user's wishes, instructions, or preferences related to events or handling at or after the end of the user's life.

The term “testamentary record” refers to a document or data set expressing instructions concerning distribution of a user's property or other testamentary dispositions.

The term “user group” refers to a set of users or entities treated collectively for access-control purposes, such as family members, professionals, or caregivers, sharing common access rights to particular data.

In one embodiment, a server, a terminal, and a networked storage subsystem cooperate to implement the claimed system. The server comprises at least one processor, a main memory, a non-volatile storage unit, and a network interface. The terminal comprises at least one processor, an audio input device such as a microphone, an audio output device such as a speaker, a display unit, a local memory, and a wireless or wired communication interface. The server and the terminal are connected via a communication network such as a packet-switched data network.

The server executes multiple software modules including a speech processing module, an intent and emotion analysis module, a generative AI interface module, a planning and orchestration module, a health and behavior analytics module, and a security and access-control module. The terminal executes an input/output control module, a local feature extraction module, and a user-interface module. The server and the terminal store configuration data and historical records in a persistent data store, for example a relational database system or a document-oriented data store.

The terminal uses a microphone to convert a user's speech into an electrical signal and digitizes the signal using an analog-to-digital converter configured for a sampling rate such as 16 kHz and a quantization depth such as 16 bits. The terminal represents the digitized signal as an array of numerical samples, i.e., time-series data, in which each element corresponds to the amplitude of the voice signal at a respective time index. The terminal stores this time-series data in a circular buffer in its local memory and associates the buffer with a record containing user identification information, time information, and an initial usage-purpose tag.

The terminal optionally executes a local feature extraction algorithm. In one embodiment, the terminal calculates Mel-frequency cepstral coefficients (MFCCs), pitch contours, and energy statistics from overlapping frames of the time-series data. The terminal thereby generates a feature vector per frame consisting of, for example, 13 MFCCs, delta and delta-delta coefficients, the fundamental frequency, and log energy. The terminal normalizes these features over a sliding window to mitigate device-specific and environment-specific variations. The terminal sends the time-series data, the feature vectors, and the associated metadata to the server via the communication interface using a secure transport protocol.

The server receives the time-series data and performs a speech recognition process. In one embodiment, the server uses a conventional speech recognition engine such as a cloud-based speech-to-text API. In another embodiment, the server executes an in-house automatic speech recognition model implemented as a deep neural network, for example a transformer-based encoder-decoder architecture trained on large-scale speech corpora. The speech recognition process applies a sequence of layers (such as convolutional front-end layers, self-attention layers, and recurrent layers) to the time-series data to produce a probability distribution over symbol sequences and then decodes the most probable sequence using beam search. The server generates character information, such as UTF-8 encoded text, representing the recognized content of the voice signal.

The server stores the character information in a character information table in a database. The server associates each character information record with the relevant user identification information, a high-resolution timestamp, usage-purpose information, and an identifier of the source time-series data. The server thereby reduces redundancy by allowing subsequent modules to reuse the same text and metadata without repeating speech recognition.

The server performs an emotion analysis process using both the character information and acoustic feature values. In one embodiment, the server executes a multi-modal emotion classifier comprising two sub-networks: a text-analysis sub-network and an acoustic-analysis sub-network. The text-analysis sub-network may be implemented as a transformer encoder that maps tokenized text to an embedding and applies attention-based pooling to produce a fixed-dimension text representation. The acoustic-analysis sub-network may be implemented as a stack of convolutional and recurrent layers that map frame-level feature vectors to an aggregated acoustic representation. The server concatenates or otherwise fuses the text representation and the acoustic representation and passes the fused vector to a fully connected layer with a softmax output. The server thereby generates an emotion analysis result including an emotion label such as “anxious,” “calm,” or “fearful” and confidence scores.

The server stores the emotion analysis result in an emotion table linked to the corresponding character information record. By maintaining a unified record that binds text, audio, emotion, and metadata, the server reduces the need for repeated parsing and re-evaluation across modules, which leads to improved computational efficiency and lower latency when multiple services operate on the same interaction.

The server executes an intent and context analysis module to derive an intent determination result from the character information. In one embodiment, the server uses a text classifier model implemented as a neural network or a gradient-boosted decision tree that receives tokenized text and outputs an intent category such as “health information query,” “end-of-life record creation,” “asset or payment inquiry,” or “store inventory question.” The classifier uses features such as word n-grams, semantic embeddings, and presence of domain-specific key terms. The server combines the intent category with the emotion analysis result, user attribute information, and history information to form a context object. This context object is represented as a structured record containing fields for the user identifier, intent category, dominant emotion label, relevant past interactions, and domain-specific indicators such as recent health-state indices.

The server uses the context object to construct a prompt sentence for a generative AI model. The generative AI model is, in one embodiment, a large-scale transformer-based language model trained with supervised and reinforcement learning on diverse text data. The server formats the prompt sentence as a sequence of natural-language instructions containing explicit role and task statements, along with embedded summaries of the context object. For example, when the user asks about health, the server generates a prompt sentence such as:
“The user said: ‘Please check my health status.’ These are the last 30 days of the user's health metrics: [summary of averages and trends]. You are a digital assistant that explains health information in simple language. Analyze the metrics and produce a brief explanation and three concrete recommendations.”

When the user expresses a desire to create a will, the server generates a prompt sentence such as:
“The user said: ‘I want to create a will.’ The detected emotion is ‘anxious’. You are an assistant that guides elderly users. Explain, step by step and in simple language, how the user can start creating a will and what information is needed. Keep the tone calm and reassuring.”

When the user is concerned about spending, the server generates a prompt sentence such as:
“The user is worried about a payment and said: ‘I don't know if this payment is really necessary.’ Suggest three short, practical pieces of advice to reduce anxiety and help the user check whether the spending is within budget.”

The server sends the prompt sentence to the generative AI model via an interface module. The interface module packages the prompt sentence into a request message, transmits it over a secure network channel, and receives response information from the model. The response information includes natural-language guidance, proposed processing steps, and, in some embodiments, structured tags indicating which domain modules to invoke. The server parses the response information using predetermined patterns, delimiters, or a light-weight parser configured to recognize action descriptors. This deterministic parsing reduces the risk of ambiguous interpretation and improves reproducibility compared to ad-hoc manual use of generative models.

In one embodiment, the generative AI model internally uses a multi-layer transformer architecture with positional encodings, self-attention heads, and feed-forward layers. The model has been trained with back-propagation using a cross-entropy loss that measures prediction error on next-token prediction tasks, and optionally with a policy-gradient or other reinforcement-learning-based fine-tuning step to optimize for instruction following. During training, the model updates its weights with stochastic gradient descent or an adaptive optimizer, using mini-batches of token sequences. The model can be further adapted to the system's domain by fine-tuning on anonymized user interactions and synthetic prompts, with data augmentation methods such as paraphrasing and controlled noise injection. By explicitly designing the prompt sentence and parsing rules, the server constrains the generative AI model to operate as a structured planning and guidance component, thereby leveraging its generalization ability without losing control over downstream actions.

The server executes a planning and orchestration module that maps the parsed response information to internal actions. For example, when the response indicates that health metrics should be analyzed, the server calls the health and behavior analytics module and passes references to relevant health-related data. When the response indicates that a will template should be created, the server calls an end-of-life document module that generates a document structure with sections for heirs, assets, and special instructions. When the response indicates that an emergency situation may exist, the server prepares a message for an emergency contact process.

The health and behavior analytics module runs on the server and uses a data analysis library such as a numerical or statistical processing framework. The server loads batches of health-related data from a health data table, where such data were previously collected from biological measurement devices and behavior measurement devices through the terminal. The server aggregates time-series sensor values across predetermined time windows and computes health-state indices, such as average resting heart rate, variability measures, and activity levels.

The server applies trend-analysis algorithms, for example linear regression or moving-window anomaly detection, to identify deviations from typical patterns. The server records these indices in a health-state index table and returns a compact summary to the planning and orchestration module.

The server analyzes these indices together with emotion analysis results. When the indices show a deteriorating trend and the emotion analysis indicates persistent negative emotions, the server can change processing priorities, for example by increasing the frequency with which monitoring processes run or by generating more detailed guidance in relation to the user's condition. This coupling of sensor-driven indices and emotion-driven signals enables the server to optimize resource allocation and notification strategies beyond what a human operator could reliably perform in real time.

The server executes a security and access-control module that manages encryption and decryption of privacy-sensitive information. When the planning and orchestration module decides to store information such as health summaries, end-of-life records, or financial guidance, the server invokes an encryption sub-module. The encryption sub-module applies a symmetric encryption algorithm such as Advanced Encryption Standard (AES) to the plaintext data using a cryptographic key managed by a key-management subsystem. The encryption sub-module generates confidential data and stores it in an external storage device, such as a remote object store or database, together with metadata including a key identifier and an access-right policy identifier.

The server stores access-right information in an access-control table that specifies which user groups (for example, family members, caregivers, or professionals) can access which categories of confidential data. The server enforces access-right information when a terminal or a third-party client issues a reference request. In particular, the server authenticates the requesting entity using credentials or tokens and checks if the entity's group membership satisfies the policy for the requested confidential data. Only if authorization is successful does the server decrypt the data or provide decrypted content. This architecture reduces the number of stages in which unencrypted data is present in memory or transmitted over the network, thus improving confidentiality.

The server adjusts the mode of presentation of information to the user based on the emotion analysis result. When the emotion analysis indicates anxiety or confusion, the server instructs the terminal to use simpler language and more step-by-step displays. When the emotion analysis indicates an emergency condition such as fear combined with certain key phrases, the server increases priority of the emergency contact process. In the emergency contact process, the server composes a notification containing the user identifier, the latest emotion label, the last recognized utterance, and location data received from the terminal. The server sends this notification via a messaging or telephony interface to pre-registered contacts. The server thereby causes a tangible interaction with external devices and communication systems, not limited to abstract data processing.

The terminal presents guidance and analysis results to the user by rendering text on the display and, in some embodiments, by playing synthetic speech. The terminal may request text-to-speech conversion from a speech synthesis engine, which transforms the character information into an audio stream that can be reproduced by the speaker. For store information support, the server can generate short, spoken-style answers and pass them to a text-to-speech engine, allowing the terminal to output audible responses through an earpiece, thereby supporting hands-free interaction in a physical environment such as a retail store.

The system improves computer technology in several ways. First, the combination of unified data structures for character information, emotion results, and context, together with a structured prompt-sentence generation mechanism, reduces redundant parsing and re-encoding of data across modules. This reduction in duplication lowers the number of memory accesses and network calls necessary to service a user request, thus reducing latency. Second, the multi-modal emotion classifier that fuses text and acoustic features yields more accurate emotion labels than prior systems that rely solely on one modality. This improved accuracy allows the server to set thresholds and trigger conditions more precisely, reducing false alarms in emergency detection and avoiding unnecessary high-priority processing. Third, the planning and orchestration module uses the generative AI model not as a simple text generator but as a structured planner constrained by explicit prompts and parsing rules. This design enables the system to automatically derive and update complex processing flows that would be impractical to encode solely as static, human-written rules, while still ensuring deterministic mapping from AI outputs to system actions.

Fourth, by integrating encryption, access-control, and emotion-aware routing into a single orchestrated pipeline, the server ensures that privacy-sensitive information is encrypted as close as possible to its point of generation and is shared only under conditions consistent with both user intent and emotional criticality. This arrangement reduces the number of intermediate storage locations that must be secured and thereby reduces the attack surface of the system. Fifth, the system is configured so that the generative AI model operates on structured context objects and well-defined prompt sentences, in contrast to generic manual prompting. The server thereby offloads from human developers a complex set of mapping rules while still achieving predictable and verifiable behavior, which constitutes a technical improvement in the design and operation of AI-based control systems.

In alternative embodiments, the server may perform more or fewer stages of processing, and certain steps may be shifted between the server and the terminal. For example, the terminal can perform part of the speech recognition process locally using an on-device neural network model optimized for low power consumption, sending only character information and emotion features to the server. This variant reduces network bandwidth usage and latency for short commands, and the server still manages the higher-level planning and security functions. In another embodiment, the emotion analysis may be simplified to use only acoustic features without text, when the system operates in noisy environments where text recognition quality is lower; in such a case, the classifier architecture and thresholds can be adjusted to maintain adequate performance while reducing computational load.

The user can employ the system in different domains without reconfiguring low-level components, because the system's core modules interpret intent and emotion in a domain-agnostic manner and generate prompt sentences that instruct the generative AI model how to tailor its behavior to specific uses. For example, the same architecture can support health information processing, end-of-life record support, and store information support, each realized by different sub-modules and context features but driven by the same generative AI interface and planning logic. As interactions accumulate, the system can further refine thresholds, model weights, and data-aggregation strategies to improve response speed and accuracy over time.

Through these concrete hardware and software configurations, data structures, and algorithmic flows, the server, terminal, and user together implement the claimed system in a manner that goes beyond an abstract idea or simple automation of manual tasks. The described arrangements improve how computer resources are organized, how multi-modal data is processed, how AI models are controlled, and how secure storage and access-control are enforced, resulting in measurable improvements in processing speed, classification accuracy, data protection, and responsiveness to emotionally critical situations.

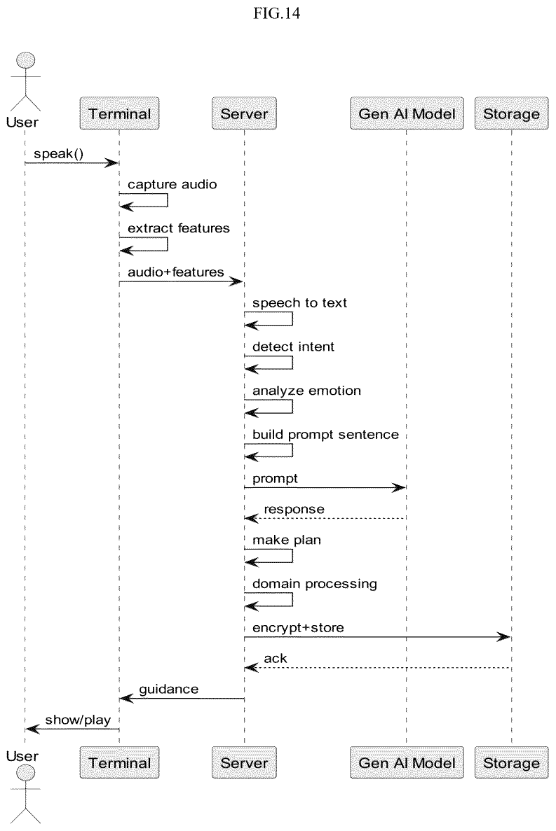

The following describes the processing flow using FIG. 14.

### Step 1:

User speaks into the terminal.

User provides spoken input, such as “Please check my health status,” “I want to create a will,” or “Is this product in stock?”
Input: Analog voice sound produced by the user.
Output: None (human speech in the physical world).

### Step 2:

Terminal captures and digitizes the voice signal.

Terminal uses a microphone to convert the analog sound into an electrical signal, samples the signal at a fixed sampling rate (e.g., 16 kHz, 16-bit), and stores the samples as time-series data in a memory buffer. Terminal attaches metadata including a temporary user ID, a timestamp, and a tentative usage-purpose tag (for example, “health,” “finance,” or “store”).
Input: Analog audio waveform at the microphone.
Output: Digital time-series data representing the voice signal and associated metadata.

### Step 3:

Terminal extracts acoustic features for emotion analysis.

Terminal segments the time-series data into overlapping frames (for example, 25 ms frames with 10 ms shift) and computes acoustic feature values, such as Mel-frequency cepstral coefficients, delta coefficients, pitch, and energy for each frame. Terminal normalizes these feature vectors (for example, mean-variance normalization) to reduce device and noise variability.
Input: Time-series audio samples from Step 2.
Output: A sequence of normalized feature vectors representing frame-level acoustic characteristics.

### Step 4:

Terminal transmits audio data, features, and metadata to the server.

Terminal packages the time-series data, feature vectors, and metadata into a message and sends the message to the server over a secure channel, such as HTTPS. Terminal may compress the audio to reduce bandwidth.
Input: Time-series data, feature vectors, and metadata from Steps 2 and 3.
Output: Network message delivered to the server containing audio data, acoustic features, and metadata.

### Step 5:

Server performs speech recognition and generates character information.

Server receives the time-series data and applies a speech recognition process. Server either calls a speech-to-text API or runs a local automatic speech recognition model that converts the waveform into a sequence of characters or words using acoustic modeling and decoding. Server also uses metadata (language, device type) to select recognition parameters.
Input: Time-series audio data and metadata from Step 4.
Output: Character information (text) representing the recognized utterance and recognition confidence scores.

### Step 6:

Server stores character information with unified metadata.

Server creates a character record in a database, storing the text, user identification information, timestamp, usage-purpose information, and a pointer to the corresponding audio data. Server assigns a unique interaction ID for later reference by other modules.
Input: Character information and metadata from Step 5.
Output: A stored character record indexed by interaction ID in the database.

### Step 7:

Server performs multi-modal emotion analysis.

Server processes both the acoustic feature vectors and the character information through an emotion classification model. Server feeds text tokens into a text sub-network and feature vectors into an acoustic sub-network, fuses the resulting embeddings, and applies a classifier layer to determine an emotion label (such as “anxious,” “calm,” or “fearful”) and corresponding confidence values.
Input: Acoustic feature vectors from Step 4 and character information from Step 5.
Output: Emotion analysis result containing at least one emotion label and confidence scores.

### Step 8:

Server stores the emotion analysis result linked to the interaction.

Server writes an emotion record into an emotion table, including the interaction ID, the emotion label, confidence, and any additional statistics (for example, valence and arousal scores). Server links this record to the character record for the same interaction.
Input: Emotion analysis result from Step 7 and interaction ID from Step 6.
Output: A stored emotion record associated with the corresponding character record.

### Step 9:

Server determines user intent category.

Server applies an intent classifier to the character information. Server tokenizes the text, extracts linguistic features, and feeds them into a classifier model that outputs an intent category such as “health information query,” “end-of-life record creation,” “asset or payment inquiry,” or “store inventory query.”
Input: Character information from Step 5.
Output: Intent determination result indicating a specific intent category and optionally a confidence score.

### Step 10:

Server constructs a context object for planning.

Server builds a structured context object that aggregates the interaction ID, user identification, character information, intent category, emotion analysis result, relevant user attributes, and selected history entries (such as recent health-state indices or past financial advice). Server organizes this context as a record or structured data type with labeled fields to enable downstream processing without repeated lookup.
Input: Intent determination result from Step 9, emotion analysis result from Step 7, character record from Step 6, and user profile and history from storage.
Output: A context object representing the current interaction and related user information.

### Step 11:

Server generates a prompt sentence for a generative AI model.

Server converts the context object into a prompt sentence by embedding the user's original text, intent description, emotion label, and a summary of relevant domain data into a natural-language instruction. For example, when intent is a health query, server composes a prompt including a health-data summary; when intent is will creation, server adds emotional state and domain guidance.
Input: Context object from Step 10.
Output: A structured prompt sentence suitable as input to a generative AI model.

### Step 12:

Server requests guidance from the generative AI model.

Server sends the prompt sentence to a generative information processing model via an API call. Server specifies generation parameters (such as maximum length or temperature) and waits for the response. Server thereby obtains response information that may include explanation text, lists of recommended actions, or structured tags.
Input: Prompt sentence from Step 11.
Output: Response information generated by the generative AI model based on the prompt.

### Step 13:

Server parses the response information and derives a processing plan.

Server analyzes the response information using predefined rules, separators, or simple parsing logic to identify instructions indicating which domain modules to call and what parameters to use. Server extracts action types (for example, “analyze health trend,” “create will template,” “send emergency alert,” or “query inventory”) and associated arguments, forming a processing plan that is represented as a list or graph of actions.
Input: Response information from Step 12.
Output: A processing plan describing concrete operations to perform within the system.

### Step 14:

Server executes domain-specific processing based on the plan.

Server reads each action in the processing plan and invokes the corresponding internal module. For health-related actions, server loads health-related data, applies aggregation and trend analysis algorithms, and computes health-state indices. For end-of-life document actions, server constructs or updates a document structure. For store-information actions, server formulates and executes database queries against product or inventory tables.
Input: Processing plan from Step 13 and domain data (such as health data, asset data, or product data) from storage.
Output: Domain-specific results, such as health summaries, document structures, inventory availability, or advisory messages.

### Step 15:

Server generates privacy-sensitive content and encrypts it.

Server identifies content in the domain-specific results that is privacy-sensitive, such as health summaries, will drafts, or financial calculations. Server applies an encryption algorithm using a cryptographic key to transform the plaintext into cipher text and forms confidential data. Server associates the confidential data with an access-right policy and a storage location identifier.
Input: Domain-specific results from Step 14 and encryption keys from a key-management system.
Output: Confidential data records and corresponding access-right and storage metadata.

### Step 16:

Server stores confidential data in external storage with access control.

Server writes the confidential data to an external storage device, such as a remote object store or database, and creates an index entry in an access-control table defining which user groups may retrieve or decrypt the data. Server links the index entry to the interaction ID and user ID to support later access requests.
Input: Confidential data and access-right metadata from Step 15.
Output: Persistently stored encrypted records and updated access-control entries.

### Step 17:

Server prepares user-oriented guidance and presentation parameters.

Server combines the domain-specific results, response information from the generative AI model, and emotion analysis result to construct user-facing messages and presentation parameters.

Server decides, based on emotion and intent, the level of detail, tone, language complexity, and whether to include additional supportive content such as relaxation advice.
Input: Domain-specific results from Step 14, emotion analysis result from Step 7, and response information from Step 12.
Output: Presentation package including text messages, optional audio messages, and display style parameters.

### Step 18:

Server optionally triggers emergency contact or monitoring escalation.

Server evaluates the combination of intent category, emotion label, and text content against predefined conditions. If an emergency is detected, server constructs an alert message containing user ID, latest utterance, emotion label, and location data if available, and sends this alert through a communication interface to registered emergency contacts.
Input: Context object from Step 10 and emotion analysis result from Step 7.
Output: Emergency notification messages sent to external recipients and log entries documenting the escalation.

### Step 19:

Server sends guidance and presentation data to the terminal.

Server packages the presentation package into a response message, optionally requesting text-to-speech synthesis by specifying which messages should be spoken aloud. Server transmits this response message to the terminal via the network.
Input: Presentation package from Step 17 and any synthesized audio or audio instructions.
Output: Network message delivered to the terminal containing guidance text, audio data or references, and display parameters.

### Step 20:

Terminal presents information to the user and controls output devices.

Terminal receives the response message and decodes its contents. Terminal displays the text guidance on the screen according to the provided layout parameters and, if requested, plays audio through the speaker using local or remote text-to-speech output. Terminal may also render controls that allow the user to confirm, correct, or proceed with further steps, such as answering questions for a will template or confirming a health summary.
Input: Response message from Step 19.
Output: Visual and/or auditory information presented to the user and updated interaction state at the terminal.

### Step 21:

User provides additional input or confirmation.

User reads or listens to the guidance and interacts with the terminal by speaking or touching the screen. User may correct misrecognized text, answer structured questions (for example, about heirs or assets), authorize sharing with family, or cancel an emergency alert.
Input: Visual and auditory information from Step 20.
Output: New spoken or typed input, selections, or confirmations that can be captured and processed starting again from Step 2 or a later step as appropriate.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

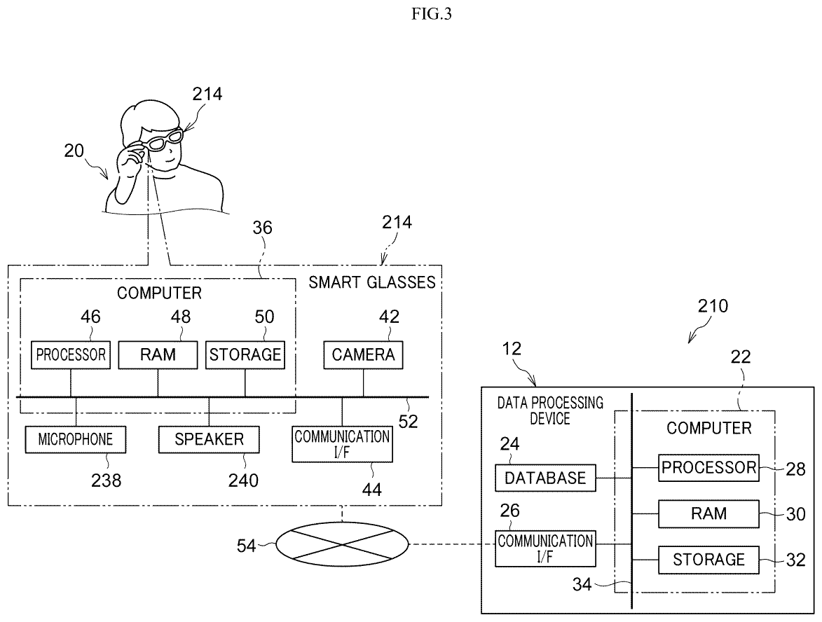

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

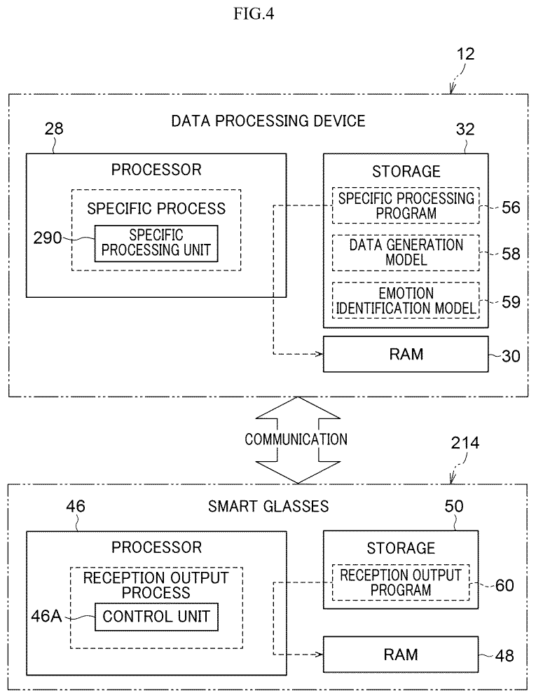

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

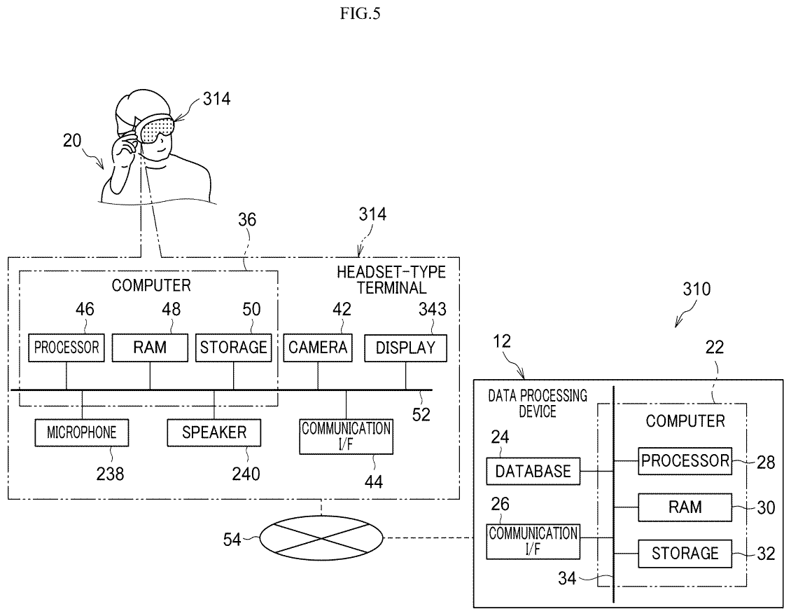

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

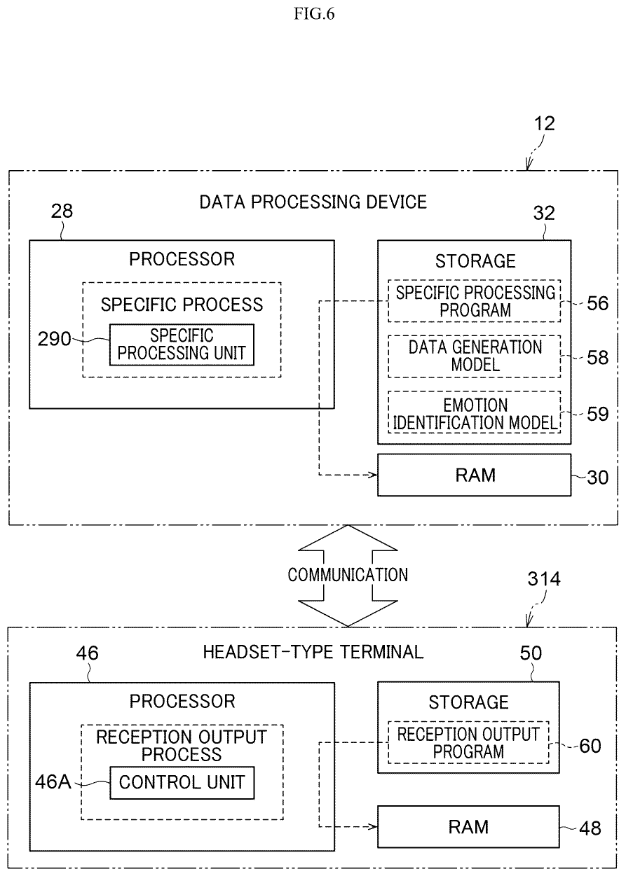

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

Fourth Exemplary Embodiment

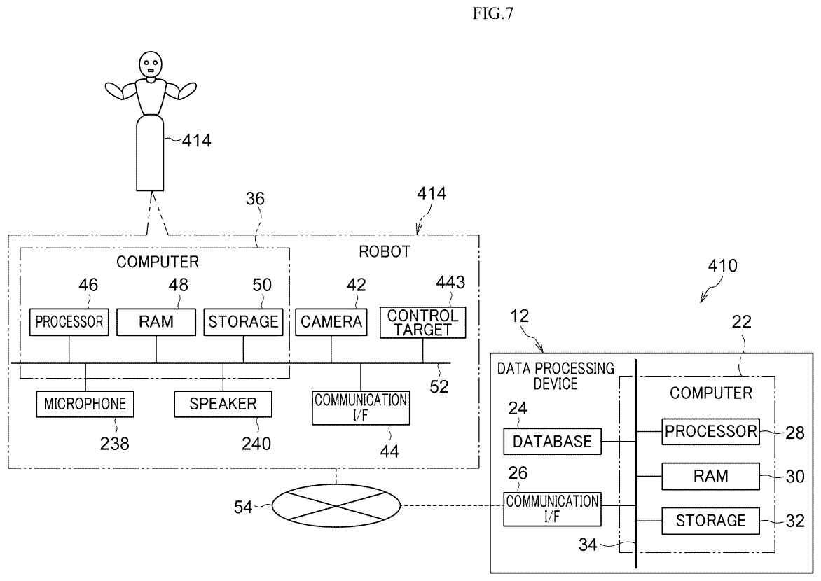

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

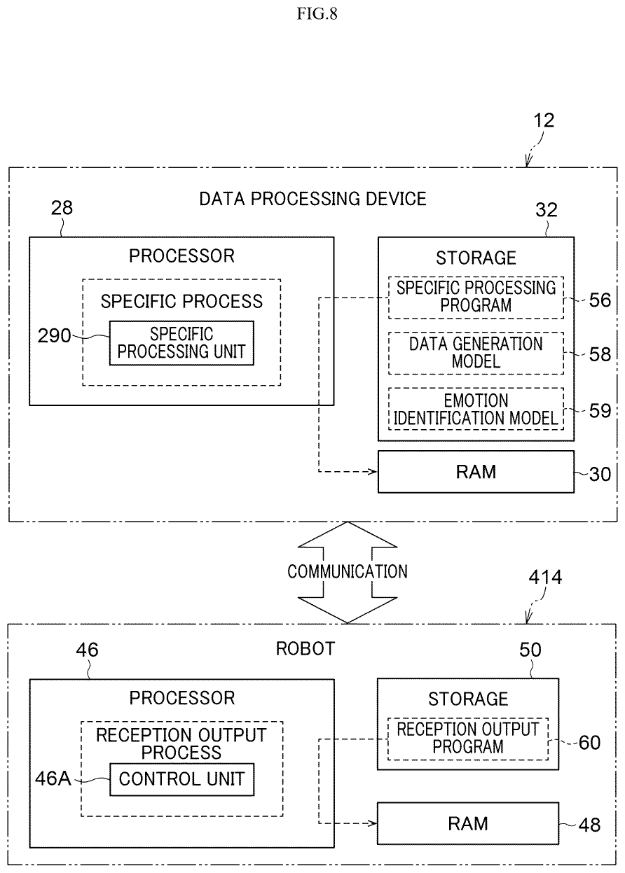

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL:https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

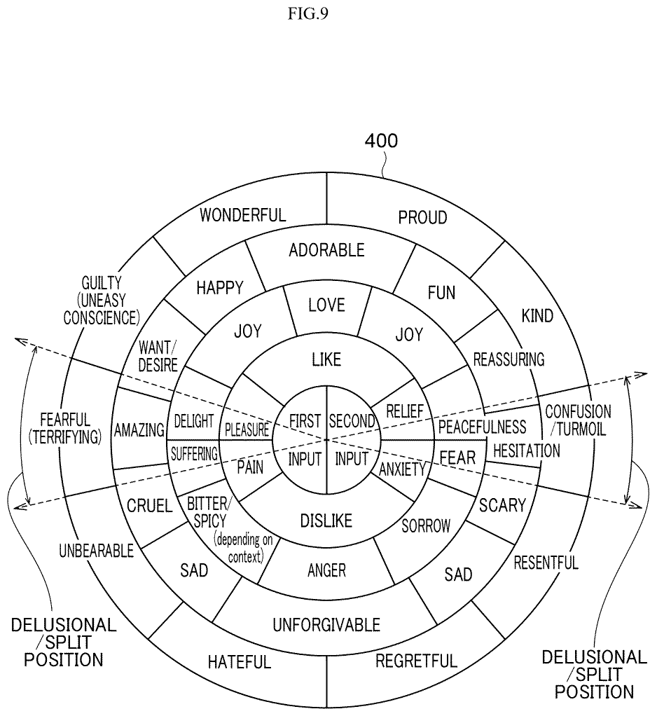

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states.

Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

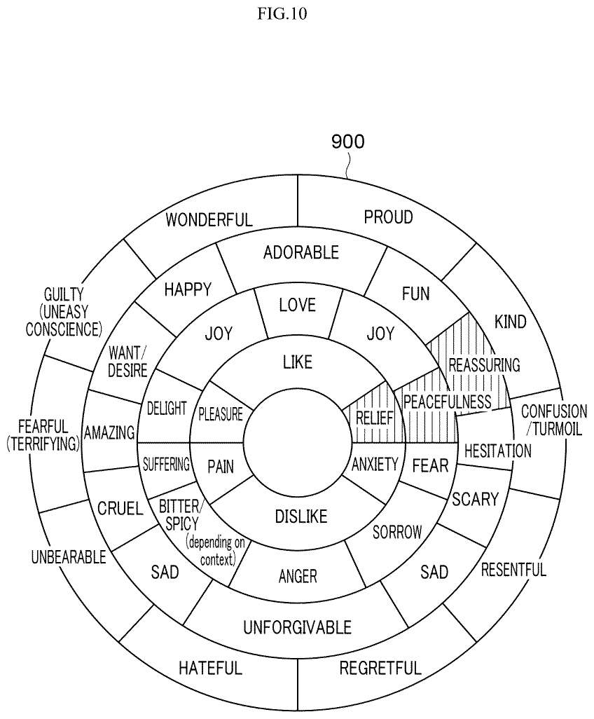

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
acquire an acoustic signal of a user by using a voice acquisition device, convert the acquired acoustic signal into digital data in a time domain or a frequency domain by using a signal processing function of an information processing apparatus, and convert the digital data into character information by inputting the digital data to a speech recognition function, input the character information to a natural language processing function and perform morphological analysis, syntactic analysis, and semantic analysis on the character information to extract request information and intent information indicating a request content and an intention of the user,
generate a prompt sentence to be given to a generative information processing model on a basis of input information including the intent information and the character information, input the prompt sentence to the generative information processing model and obtain, from the generative information processing model, processing content or output information corresponding to the intention of the user,
select, on a basis of the intent information, at least one of an external information acquisition function and a state information acquisition function that is configured to acquire user state information or external environment information, and access an external information processing service or a state information management service via a network to acquire the user state information or the external environment information,
perform at least one of an aggregation process, a comparison process, and an evaluation process on the acquired user state information or the external environment information, and combine a result of the at least one process with the output information obtained from the generative information processing model to generate service content or proposal content to be presented to the user,
extract acoustic feature values from the acoustic signal, input the acoustic feature values to an emotion estimation model to estimate an emotional state of the user, and change at least one of an expression form and a providing method of the prompt sentence, the service content, or the proposal content in accordance with the emotional state, and
present the service content or the proposal content to the user by using a display device or an audio output device, and acquire, by the voice acquisition device, additional voice input from the user in response to the presentation and repeatedly perform interactive processing on a basis of the additional voice input.

### Supplementary 2

The system according to supplementary 1,
wherein the processor is configured to
use, as the state information acquisition function, biological activity information obtained from a biological activity information management service or a biological information management function, the biological activity information including at least one of activity amount information, heart rate information, and sleep information, evaluate a health state of the user on a basis of the biological activity information, include a result of the evaluation in the prompt sentence to be input to the generative information processing model, and generate, as the service content, at least one of an explanatory text, an advice text, and a goal setting content relating to health management.

### Supplementary 3

The system according to supplementary 1,
wherein the processor is configured to
use, as the external information acquisition function, document template information and asset information, specify a document type and an asset type from the character information of the user by using the natural language processing function, input, to the generative information processing model, a prompt sentence including a result of the specification and the document template information, generate, on a basis of output from the generative information processing model, a draft of a record document or an expression-of-intention document relating to end-of-life preparation, and manage the asset information of the user and desired contents of the user on a basis of the draft.

### Application Example 1

### Supplementary 1

A system comprising a processor,
wherein the processor is configured to
acquire a voice signal of a user by using a voice input device, convert the acquired voice signal into character information by using a speech recognition technique,
analyze input information including the character information and user attribute information to determine an intention of the user and a requested domain,
generate, based on a result of the determination, processing request information representing at least one of product information provision, health condition support, asset condition support, and document creation support, the processing request information including search condition information or state evaluation information,
search, based on the search condition information, a product information storage device, and generate structured data by using product information acquired as a search result,
acquire, based on the state evaluation information, related information from at least one of a health information storage device and an asset information storage device, and generate structured data by using the acquired information,
generate a prompt sentence including the character information and the structured data, and input the prompt sentence into a generative AI model to cause the generative AI model to generate explanation information or recommendation information,
integrate the generated explanation information or recommendation information with the structured data to generate response information to be presented to the user,
extract features from the voice signal of the user and estimate an emotional state of the user based on the extracted features,
dynamically adjust at least one of contents of the prompt sentence, an expression style of the explanation information or the recommendation information, and a presentation method of the response information, in accordance with the emotional state and the requested domain,
acquire, when the system is used in a physical store, location information associated with the product information, and add guidance information for store navigation based on the location information to the response information, and
repeat the above processing in response to additional voice input from the user, and update the prompt sentence and the structured data in consideration of a dialogue history.

### Supplementary 2

The system according to supplementary 1,
wherein the processor is configured to
accumulate biometric information and behavior information acquired from the user in a storage device, calculate a health index as the state evaluation information, include the health index in the prompt sentence to be input into the generative AI model, cause the generative AI model to generate lifestyle guidance information as the explanation information or the recommendation information based on the health index, and provide the lifestyle guidance information as part of the response information.

### Supplementary 3

The system according to supplementary 1,
wherein the processor is configured to
generate document candidate information by assigning family structure information, wish information, and asset-related information acquired from the user to a document template, include the document candidate information in the prompt sentence to be input into the generative AI model, cause the generative AI model to generate, as the explanation information, a draft text for at least one of an end-of-life record document and an intention expression document, provide the draft text as the response information in an editable format, and integrally store and update the asset-related information as asset management information including digital assets.

### Example 2

### Supplementary 1

A system comprising a processor,
wherein the processor is configured to
acquire audio data representing a user's voice by using an acoustic input/output device for capturing audio information of the user,
convert the acquired audio data into character information by using a speech recognition algorithm,
construct a prompt sentence serving as input to a generative AI model, based on the converted character information and template information corresponding to a function type,
input the constructed prompt sentence into the generative AI model and cause the generative AI model to generate document data including at least one of a report document, a record document, and an instruction document corresponding to the character information,
perform formatting processing and additional-information attaching processing on the generated document data to generate file data in a predetermined electronic document format,
upload the generated file data to an external storage service and generate shared link information for accessing the file data,
notify the shared link information to an information processing terminal of the user and enable the shared link information to be transmitted to another party via the information processing terminal,
reconstruct, based on additional input from the user regarding the audio data or the character information, a prompt sentence for instructing modification or addition with respect to existing document data, and regenerate updated document data by using the generative AI model,
extract time-domain feature quantities and frequency-domain feature quantities from the audio data and estimate an emotional state of the user by using an emotion estimation model,
dynamically adjust at least one of an instruction content, a writing style, and an output format of the prompt sentence based on the estimated emotional state and user attribute information, and
change at least one of content and expression manner of the generated document data, and perform an arithmetic processing for aggregating stored health-related data or asset-related data, generate an information-provision prompt sentence based on an aggregation result, and cause the generative AI model to generate at least one of an explanatory document and a summary document.

### Supplementary 2

The system according to supplementary 1,
wherein the processor is configured to
store biological information or life-log information of the user in at least one of the external storage service and an internal storage device in a time series, calculate a trend index of a health condition by aggregating the biological information or the life-log information over a predetermined period, generate a health-condition explanation prompt sentence based on the calculated trend index and the biological information or the life-log information, and cause the generative AI model to generate a health-condition report document.

### Supplementary 3

The system according to supplementary 1,
wherein the processor is configured to
store asset information and inheritance-preference information of the user as character information, generate asset list data and valuation data by classifying and evaluating the stored asset information, generate a prompt sentence including an instruction for creating at least one of an ending-related document and a will-related document based on the asset list data and the inheritance-preference information, cause the generative AI model to generate a draft of the at least one of the ending-related document and the will-related document, store the generated draft in the external storage service, and enable sharing of the generated draft with a specialist or a related person by using the shared link information.

### Application Example 2

### Supplementary 1

A system comprising a processor,
wherein the processor is configured to
acquire a voice signal of a user from an input device configured to capture user speech and encode the voice signal as time-series data,
convert the time-series data into character information by applying a speech recognition process, and store the character information in association with user identification information, time information, and usage-purpose information,
extract acoustic feature values from the voice signal or the time-series data, generate an emotion analysis result by classifying an emotional state of the user based on the character information and the acoustic feature values, and store the emotion analysis result in association with the character information,
derive an intent determination result indicating an intent category from the character information, and construct a prompt sentence for input to a generative information processing model based on the intent determination result, the emotion analysis result, and attribute information or history information relating to the user,
input the prompt sentence to the generative information processing model, obtain response information from the generative information processing model, analyze the response information,
and identify a processing plan or guidance information relating to at least one of health information processing, asset information processing, life information processing, end-of-life record support, and store information support,
adjust content or a mode of presentation of information to the user based on the processing plan or the guidance information and the emotion analysis result, and execute, as needed, at least one of an emergency contact process, a monitoring process, and a psychological burden-reduction process,
generate confidential data by subjecting privacy-sensitive information, which is generated based on the character information or the processing plan, to an encryption process, store the confidential data in an external storage device, and manage access-right information for the confidential data on a per-user-group basis, and
perform authentication and authorization for a reference request to the confidential data based on the access-right information, and provide the confidential data or information decrypted from the confidential data to a terminal device having authorization.

### Supplementary 2

The system according to supplementary 1,
wherein the processor is configured to
accumulate health-related data acquired from a biological measurement device or a behavior measurement device, calculate a health-state index by statistical processing or trend-analysis processing, generate health-management information based on the health-state index and the response information from the generative information processing model, and provide the health-management information to the user or a related person in an expression adapted to the emotion analysis result.

### Supplementary 3

The system according to supplementary 1,
wherein the processor is configured to
acquire, by using the speech recognition process and the generative information processing model, intention content, property information, and family-structure information of the user, automatically generate a document structure for an end-of-life record or a testamentary record based on an acquisition result, progressively complete the document structure while varying at least one of a question order, explanation content, and auxiliary information in accordance with the emotion analysis result, and store and manage the completed document structure and
associated asset information so as to be storable and shareable based on the encryption process and the access-right information.

What is claimed is:

1. A system comprising:
circuitry configured to:
acquire, via an audio acquisition device coupled to a terminal device, an acoustic signal of a user, and convert the acoustic signal into digital data by a signal processing function;
convert the digital data into character information by inputting the digital data to a speech recognition function operating on a neural network model;
input the character information to a natural language processing function and perform at least morphological analysis, syntactic analysis, and semantic analysis on the character information to extract intent information indicating an intention of the user;
generate, based on input information including the intent information and the character information, a prompt sentence for a generative neural network model operating on a deep learning framework;
input the prompt sentence to the generative neural network model and acquire output information corresponding to the intention of the user;
extract acoustic feature values from the acoustic signal and input the acoustic feature values to an emotion estimation model to estimate an emotional state of the user; and
adjust at least one of an expression form and a providing method of content to be presented to the user in accordance with the estimated emotional state, and transmit the content to the terminal device via a communication interface coupled to a packet-switched network.

2. The system according to claim 1, wherein the circuitry is configured to select, based on the intent information, at least one of an external information acquisition function and a state information acquisition function, and access an external information processing service or a state information management service via the packet-switched network to acquire user state information or external environment information.

3. The system according to claim 2, wherein the circuitry is configured to perform at least one of an aggregation process, a comparison process, and an evaluation process on the acquired user state information or external environment information, and combine a result of the at least one process with the output information obtained from the generative neural network model to generate service content or proposal content to be presented to the user.

4. The system according to claim 3, wherein the circuitry is configured to present the service content or the proposal content to the user by using a display device or an audio output device of the terminal device, and acquire, by the audio acquisition device, additional voice input from the user in response to the presentation and repeatedly perform interactive processing on a basis of the additional voice input.

5. The system according to claim 4, wherein the user state information includes biological activity information obtained from a biological activity information management service, the biological activity information including at least one of activity amount information, heart rate information, and sleep information, and the circuitry is configured to evaluate a health state of the user on a basis of the biological activity information and include a result of the evaluation in the prompt sentence to generate health management guidance as the service content.

6. The system according to claim 5, wherein the circuitry is configured to accumulate biological information and behavior information acquired from the user in a storage device in a time series, calculate a trend index of a health condition by aggregating the biological information over a predetermined period, and generate a health condition report document by including the trend index in the prompt sentence input to the generative neural network model.

7. The system according to claim 6, wherein the circuitry is configured to execute, based on the estimated emotional state, at least one of an emergency contact process when the emotional state indicates acute distress, a monitoring process for continuous observation, and a psychological burden reduction process that adjusts complexity of information presented to the user.

8. The system according to claim 1, wherein the circuitry is configured to construct the prompt sentence based on the character information and template information corresponding to a function type indicated by the intent information, and cause the generative neural network model to generate document data including at least one of a report document, a record document, and an instruction document.

9. The system according to claim 8, wherein the circuitry is configured to perform formatting processing and additional-information attaching processing on the generated document data to generate file data in a predetermined electronic document format, upload the file data to an external storage service via the communication interface, and generate shared link information for accessing the file data.

10. The system according to claim 9, wherein the circuitry is configured to reconstruct, based on additional input from the user regarding the character information, a further prompt sentence for instructing modification or addition with respect to the document data, and regenerate updated document data by using the generative neural network model.

11. The system according to claim 10, wherein the document data includes a draft of a record document or an expression-of-intention document relating to end-of-life preparation, the circuitry is configured to manage asset information of the user including digital assets based on the draft, and the shared link information enables sharing of the draft with a designated recipient.

12. The system according to claim 1, wherein the circuitry is configured to analyze the character information and user attribute information to determine a requested domain, generate processing request information representing at least one of information provision, state support, and creation support, and search a data storage device based on search condition information derived from the processing request information.

13. The system according to claim 12, wherein the circuitry is configured to generate structured data by using information acquired as a search result, generate the prompt sentence including the character information and the structured data, and input the prompt sentence to the generative neural network model to cause the generative neural network model to generate explanation information or recommendation information, and integrate the generated explanation information or recommendation information with the structured data to generate response information.

14. The system according to claim 13, wherein the circuitry is configured to acquire location information associated with product information when the system is used in a physical store environment, add guidance information for store navigation based on the location information to the response information, and repeat the interactive processing in response to additional voice input while updating the prompt sentence and the structured data in consideration of a dialogue history.

15. The system according to claim 1, wherein the acoustic feature values include at least pitch, intensity, spectral characteristics, and temporal variations extracted from the acoustic signal, and the emotion estimation model comprises a neural network trained on labeled audio datasets to output a probability distribution over a plurality of discrete emotion classes.

16. The system according to claim 15, wherein the circuitry is configured to derive the intent information as an intent category from the character information, construct the prompt sentence based on the intent category, the estimated emotional state, and attribute information or history information relating to the user, and identify from the output information a processing plan relating to at least one of information processing, document creation support, and state monitoring.

17. The system according to claim 16, wherein the circuitry is configured to generate confidential data by subjecting privacy-sensitive information generated based on the character information or the processing plan to an encryption process, store the confidential data in an external storage device, manage access-right information for the confidential data on a per-user-group basis, and perform authentication and authorization for a reference request to the confidential data based on the access-right information.

18. A system comprising:
circuitry configured to:
acquire, via an audio acquisition device coupled to a terminal device, an acoustic signal of a user, convert the acoustic signal into digital data in a time domain and a frequency domain by a signal processing function, and convert the digital data into character information by inputting the digital data to a speech recognition function that uses an acoustic model and a language model;
input the character information to a natural language processing function and perform morphological analysis, syntactic analysis, and semantic analysis on the character information to extract request information and intent information indicating a request content and an intention of the user;
generate, based on input information including the intent information, the character information, and user attribute information, a prompt sentence for a transformer-based generative neural network model comprising a plurality of self-attention layers and feed-forward layers;
input the prompt sentence to the transformer-based generative neural network model and acquire output information corresponding to the intention of the user;
select, based on the intent information, at least one of an external information acquisition function and a state information acquisition function, and access via a packet-switched network an external information processing service to acquire user state information, and perform at least one of an aggregation process and an evaluation process on the acquired user state information and combine a result with the output information to generate service content;
extract acoustic feature values including pitch, intensity, spectral characteristics, and temporal variations from the acoustic signal, and input the acoustic feature values to an emotion estimation model comprising a neural network to estimate an emotional state of the user; and
adjust at least one of an expression form, a detail level, and a providing method of the service content in accordance with the estimated emotional state, and transmit the service content to the terminal device via a communication interface for presentation by a display device or an audio output device.

19. The system according to claim 18, wherein the emotion estimation model is trained using supervised learning on labeled audio datasets by minimizing a cross-entropy loss between predicted emotion class distributions and reference emotion labels, and the circuitry is configured to fuse an output of the emotion estimation model with a sentiment analysis result derived from the character information by a weighted combination to produce a multi-modal emotional state estimate.

20. A method performed by circuitry of a system coupled to a packet-switched network via a communication interface, the method comprising:
acquiring, via an audio acquisition device coupled to a terminal device, an acoustic signal of a user, and converting the acoustic signal into digital data by a signal processing function;
converting the digital data into character information by inputting the digital data to a speech recognition function operating on a neural network model;
inputting the character information to a natural language processing function and performing at least morphological analysis, syntactic analysis, and semantic analysis on the character information to extract intent information indicating an intention of the user;
generating, based on input information including the intent information and the character information, a prompt sentence for a generative neural network model operating on a deep learning framework;
inputting the prompt sentence to the generative neural network model and acquiring output information corresponding to the intention of the user;
extracting acoustic feature values from the acoustic signal and inputting the acoustic feature values to an emotion estimation model to estimate an emotional state of the user; and
adjusting at least one of an expression form and a providing method of content to be presented to the user in accordance with the estimated emotional state, and transmitting the content to the terminal device via the communication interface.
